SEQUENCE LISTING (40 sequences)

SEQ ID NO: 1 - protein, 21 aa, Artificial Sequence
  Description of Artificial Sequence Synthetic peptide
  MISC_FEATURE (1)..(3): May or may not be present
GSGEGRGSLLTCGDVEENPGP

SEQ ID NO: 2 - protein, 22 aa, Artificial Sequence
  Description of Artificial Sequence Synthetic peptide
  MISC_FEATURE (1)..(3): May or may not be present
GSGATNFSLLKQAGDVEENPGP

SEQ ID NO: 3 - protein, 23 aa, Artificial Sequence
  Description of Artificial Sequence Synthetic peptide
  MISC_FEATURE (1)..(3): May or may not be present
GSGQCTNYALLKLAGDVESNPGP

SEQ ID NO: 4 - protein, 25 aa, Artificial Sequence
  Description of Artificial Sequence Synthetic peptide
  MISC_FEATURE (1)..(3): May or may not be present
GSGVKQTLNFDLLKLAGDVESNPGP

SEQ ID NO: 5 - DNA, 25 bp, Mus sp.
gaaaggtttattgtaagaaagccaa

SEQ ID NO: 6 - DNA, 13 bp, Mus sp.
gaaaggtttattg

SEQ ID NO: 7 - DNA, 16 bp, Mus sp.
gaaaggtttattgtaa

SEQ ID NO: 8 - DNA, 10 bp, Mus sp.
gaaaggttta

SEQ ID NO: 9 - DNA, 16 bp, Mus sp.
gaaaggtttattgaag

SEQ ID NO: 10 - DNA, 20 bp, Mus sp.
gaaaggtttattgtaagaaa

SEQ ID NO: 11 - DNA, 25 bp, Mus sp.
gaaaggtttattgtaagaaagccaa

SEQ ID NO: 12 - DNA, 24 bp, Mus sp.
gaaaggtttattgtaagaaagcca

SEQ ID NO: 13 - DNA, 17 bp, Mus sp.
gaaaggtttattgtaag

SEQ ID NO: 14 - DNA, 50 bp, Artificial Sequence
  Description of Artificial Sequence Synthetic oligonucleotide
  modified_base (1)..(2): a, c, t, g, unknown or other
nntgcgggggtggggagtggaagcgggcacctcactgggcagtcagagtc

SEQ ID NO: 15 - DNA, 34 bp, Mus sp.
gtggaagcgggcacctcactgggcagtcagagtc

SEQ ID NO: 16 - DNA, 37 bp, Mus sp.
agtggaagcgggcacctcactgggcagtcagagtctc

SEQ ID NO: 17 - DNA, 35 bp, Mus sp.
tggaagcgggcacctcactgggcagtcagagtctc

SEQ ID NO: 18 - DNA, 35 bp, Mus sp.; identical to SEQ ID NO: 17

SEQ ID NO: 19 - DNA, 38 bp, Mus sp.
gagtggaagcgggcacctcactgggcagtcagagtctc

SEQ ID NO: 20 - DNA, 47 bp, Mus sp.
gggggtggggagtggaagcgggcacctcactgggcagtcagagtctc

SEQ ID NO: 21 - DNA, 37 bp, Mus sp.; identical to SEQ ID NO: 16

SEQ ID NO: 22 - DNA, 50 bp, Mus sp.
tgcgggggtggggagtggaagcgggcacctcactgggcagtcagagtctc

SEQ ID NO: 23 - DNA, 50 bp, Artificial Sequence
  Description of Artificial Sequence Synthetic oligonucleotide
  modified_base (1)..(1): a, c, t, g, unknown or other
ntgcgggggtggggagtggaagcgggcacctcactgggcagtcagagtct

SEQ ID NO: 24 - DNA, 49 bp, Mus sp.
gcgggggtggggagtggaagcgggcacctcactgggcagtcagagtctc

SEQ ID NO: 25 - DNA, 48 bp, Mus sp.
cgggggtggggagtggaagcgggcacctcactgggcagtcagagtctc

SEQ ID NO: 26 - protein, 9 aa, Artificial Sequence
  Description of Artificial Sequence Synthetic peptide
  MOD_RES (5)..(5): Any amino acid
GDVEXNPGP

SEQ ID NO: 27 - DNA, 75 bp, Mus sp.
gaaaggtttattgtaagaaagccaatgcgggggtggggagtggaagcgggcacctcactgggcagtcagagtctc

SEQ ID NO: 28 - DNA, 75 bp, Artificial Sequence
  Description of Artificial Sequence Synthetic oligonucleotide
  modified_base (26)..(26): a, c, t, g, unknown or other
gaaaggtttattgtaagaaagccaantgcgggggtggggagtggaagcgggcacctcactgggcagtcagagtct

SEQ ID NO: 29 - DNA, 74 bp, Artificial Sequence
  Description of Artificial Sequence Synthetic oligonucleotide
gaaaggtttattgtaagaaagccaagcgggggtggggagtggaagcgggcacctcactgggcagtcagagtctc

SEQ ID NO: 30 - DNA, 73 bp, Artificial Sequence
  Description of Artificial Sequence Synthetic oligonucleotide
gaaaggtttattgtaagaaagccaacgggggtggggagtggaagcgggcacctcactgggcagtcagagtctc

SEQ ID NO: 31 - DNA, 75 bp, Artificial Sequence
  Description of Artificial Sequence Synthetic oligonucleotide
  modified_base (26)..(27): a, c, t, g, unknown or other
gaaaggtttattgtaagaaagccaanntgcgggggtggggagtggaagcgggcacctcactgggcagtcagagtc

SEQ ID NO: 32 - DNA, 47 bp, Artificial Sequence
  Description of Artificial Sequence Synthetic oligonucleotide
gaaaggtttattggtggaagcgggcacctcactgggcagtcagagtc

SEQ ID NO: 33 - DNA, 53 bp, Artificial Sequence
  Description of Artificial Sequence Synthetic oligonucleotide
gaaaggtttattgtaaagtggaagcgggcacctcactgggcagtcagagtctc

SEQ ID NO: 34 - DNA, 51 bp, Artificial Sequence
  Description of Artificial Sequence Synthetic oligonucleotide
gaaaggtttattgtaatggaagcgggcacctcactgggcagtcagagtctc

SEQ ID NO: 35 - DNA, 45 bp, Artificial Sequence
  Description of Artificial Sequence Synthetic oligonucleotide
gaaaggtttatggaagcgggcacctcactgggcagtcagagtctc

SEQ ID NO: 36 - DNA, 54 bp, Artificial Sequence
  Description of Artificial Sequence Synthetic oligonucleotide
gaaaggtttattgaaggagtggaagcgggcacctcactgggcagtcagagtctc

SEQ ID NO: 37 - DNA, 70 bp, Artificial Sequence
  Description of Artificial Sequence Synthetic oligonucleotide
gaaaggtttattgtaagaaatgcgggggtggggagtggaagcgggcacctcactgggcagtcagagtctc

SEQ ID NO: 38 - DNA, 72 bp, Artificial Sequence
  Description of Artificial Sequence Synthetic oligonucleotide
gaaaggtttattgtaagaaagccaagggggtggggagtggaagcgggcacctcactgggcagtcagagtctc

SEQ ID NO: 39 - DNA, 74 bp, Artificial Sequence
  Description of Artificial Sequence Synthetic oligonucleotide
gaaaggtttattgtaagaaagccatgcgggggtggggagtggaagcgggcacctcactgggcagtcagagtctc

SEQ ID NO: 40 - DNA, 54 bp, Artificial Sequence
  Description of Artificial Sequence Synthetic oligonucleotide
gaaaggtttattgtaagagtggaagcgggcacctcactgggcagtcagagtctc

(19) United States
(12) Patent Application Publication
(10) Pub. No.: US 2023/0001020 A1
(43) Pub. Date: Jan. 5, 2023

(54) SELECTIVE EXPANSION OF GENE-TARGETED CELLS

(71) Applicant: Baylor College of Medicine, Houston, TX (US)

(72) Inventors: William Lagor, Houston, TX (US); Ayrea Hurley, Houston, TX (US); Kelsey Jarrett, Houston, TX (US); Karl-Dimiter Bissing, Houston, TX (US); Marco De Giorgi, Houston, TX (US); Mia Furgurson, Houston, TX (US)

(73) Assignee: Baylor College of Medicine, Houston, TX (US)

(21) Appl. No.: 17/756,242

(22) PCT Filed: Nov. 20, 2020
(86) PCT No.: PCT/US20/61605

§ 371 Date: May 19, 2022

Related U.S. Application Data
(60) Provisional application No. 62/939,795, filed on Nov. 25, 2019.

(51) Int. Cl. A61K 48/00; C12N 15/11; C12N 9/22; C12N 9/10; C12N 9/04; A61P 1/16; C12N 15/86; C12N 15/113

(52) U.S. Cl. CPC A61K 48/0066; C12N 15/11; C12N 9/22; C12N 9/1085; C12Y 205/01087; C12N 9/0006; C12Y 101/01088; A61P 1/16; C12N 15/86; C12N 15/113; C12N 2310/20; C12N 2800/80; C12N 2750/14143; C12N 2750/14171; C12N 2310/14; C12N 2310/531

(57) ABSTRACT

Embodiments of the disclosure encompass systems, methods, and compositions related to selective advantages to somatic cells that harbor one or more particular genetic modifications. In particular embodiments, there is selective expansion of gene-targeted cells wherein the strategy involves deletion of an essential gene product that is replaced with targeted integration that also includes integration of a therapeutic transgene. The cells that harbor the replaced essential gene product, and thereby the therapeutic transgene, are selected for using pharmaceutical or nutritional agents that are linked to the function of the essential gene product.

### STATEMENT REGARDING FEDERALLY SPONSORED RESEARCH OR DEVELOPMENT

This invention was made with government support under HL132840 awarded by the National Institutes of Health. The government has certain rights in the invention.

This application claims priority to U.S. Provisional Patent Application Ser. No. 62/939,795, filed Nov. 25, 2019, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Nov. 19, 2020, is named BAYM_P0287WO_SL.txt and 9,483 bytes in size.

### TECHNICAL FIELD

Embodiments of the disclosure include at least the fields of cell biology, molecular biology, gene therapy, and medicine.

### BACKGROUND

Monogenic disorders of the liver are individually rare but collectively common (˜10/1000 live births)(1), and adversely impact quality of life for millions of patients worldwide. Great progress has been made in liver-directed gene therapy. In particular, Adeno-Associated Viral (AAV) vectors have been shown to be both safe and effective in Phase I/II trials to treat Hemophilia A and B(2-5). While these therapies are likely to receive regulatory approval in the coming years, achieving permanent life-long correction will be difficult. Immune responses to the AAV capsid can lead to elimination of the transduced hepatocytes by cytotoxic T-cells(4,6). Even if these T-cell responses can be managed with short-term immunosuppression, a more fundamental obstacle exists. The recombinant AAV genome is episomal (i.e., non-integrating) (7) and will be lost over time with normal hepatocyte turnover and cell division. It is estimated that the typical lifespan of a hepatocyte is between 200 and 400 days (8,9). The rate turnover may be even greater for metabolic diseases, and there are also pediatric disorders that must be treated before the liver is fully grown (10). Therefore, a truly durable liver-directed gene therapy ultimately requires permanent changes to the patient's own DNA.

### BRIEF SUMMARY

The present disclosure is directed to systems, methods, and compositions for selective expansion of gene-targeted cells. Embodiments include gene therapy for an individual in which case the cells that have the corrected gene are selectively expanded because they also have an essential gene product, which gives them a growth advantage over non-edited cells. In particular cases, the expression of the therapeutic gene is linked to expression of an essential gene product, and each are present in cells that are lacking production of the corresponding endogenous gene product. Cells in which both the exogenously provided therapeutic gene and essential gene are present are protected from external pressure from conditions for which the essential gene is required.

In certain embodiments, somatic deletion of an essential gene is performed to promote expansion of gene-edited cells, such as hepatocytes. Specific embodiments of the disclosure utilize clinically approved drugs or natural products, for example, to control selection. In specific embodiments, the essential gene is knocked down by siRNA, shRNA, anti-sense oligonucleotides, etc.

In specific embodiments of the disclosure applied to liver medical conditions, endogenously expressed enzymes are utilized for positive selection in the liver. In specific embodiments, there is provided a “scarless” approach to expand gene-corrected hepatocytes that restores activity of the endogenous enzyme used for selection, without altering any other gene related to the selection advantage (i.e. deletion of Hpd or Por). The disclosure also provides a generalizable approach for integration and expansion that is applicable to numerous liver diseases and not just those with a pre-existing advantage to corrected cells.

Embodiments of the disclosure encompass systems, comprising: (a) a first polynucleotide comprising an expression cassette, said expression cassette comprising a therapeutic polynucleotide linked to an essential gene product polynucleotide, wherein said cassette comprises one or more sequences capable of integrating at least part of the cassette at a first endogenous locus; and one of (b1) or (b2): (b1) a second polynucleotide comprising a targeting region capable of inhibiting, knocking down, or disrupting expression of the second endogenous locus and/or the activity of a gene product therefrom, (b2) a second polynucleotide comprising a targeting region that targets integration at a second endogenous locus to disrupt expression of the second endogenous locus and/or the activity of a gene product therefrom, wherein for (b1) or (b2) said second endogenous locus encodes the essential gene product. In some cases, the therapeutic polynucleotide and the essential gene product polynucleotide are linked by a means for co-expression of the therapeutic polynucleotide and the essential gene product polynucleotide. The means for co-expression comprises a 2A element or an IRES element, in at least some cases. In specific embodiments, in a 5′ to 3′ direction in the expression cassette, the therapeutic polynucleotide is 5′ or 3′ to the essential gene product polynucleotide. In specific cases, the first endogenous locus is the second endogenous locus. The essential gene product polynucleotide may be fused to the therapeutic polynucleotide.

In particular embodiments, the targeting region comprises guide RNA sequence for a CRISPR/Cas9 system or the targeting region comprises shRNA, siRNA, anti-sense oligonucleotide, locked nucleic acids, or chemically modified derivatives thereof. The first polynucleotide and/or the second polynucleotide may serve as a template of integration, in particular aspects, and the first polynucleotide and/or the second polynucleotide may be present in a vector of any kind, such as a nanoparticle, plasmid, adeno-associated viral vector, lentiviral vector, retroviral vector, or combination thereof. Any vector may be an integrating vector or a non-integrating vector. When integration occurs at the first endogenous locus, the integration may be targeted integration or random integration. Integration at the first endogenous locus may result in control of expression of the expression cassette from regulatory sequence(s) at the first endogenous locus, and in some cases the expression cassette lacks a promoter.

In particular embodiments, disruption or reduction of expression at the second endogenous locus that encodes the essential gene product, or disruption of the activity of a gene product therefrom, is therapeutically treatable by one or more nutritional or pharmacological agents to substitute for absence of the essential gene product. In specific cases, the essential gene product polynucleotide is configured to be resistant to disruption of expression by the targeting region.

In specific cases, the first endogenous locus is ApoA1 (APOA1), albumin (ALB), haptoglobin (HP), serum amyloid a1 (SAA1), orosomucoid 1 (ORM1), ferritin light chain (FTL), Apolipoprotein C3 (APOC3), fibrinogen beta chain (FGB), fibrinogen gamma chain (FGG), serpin family A member 1 (SERPINA1) or fumarylacetoacetate hydrolase (FAH). The essential gene product may be fumarylacetoacetate hydrolase (FAH), dehydrodolichyl diphosphate synthase subunit (DHDDS), or 3-hydroxy-3-methylglutaryl Co-enzyme A reductase (HMGCR), UDP glucuronosyltransferase family 1 member A1 (UGT1A1), or methylmalonyl coA mutase (MMUT). In specific embodiments, the pharmacological agent is nitisinone. In specific cases, when the essential gene product is DHDDS, cholesterol in the diet of the individual is used for negative selection pressure. When the essential gene product is HMGCR, mevalonic acid may be used for protection of hepatocytes from selection.

Any system of the disclosure may be utilized ex vivo or in vivo in a mammal, including a human, dog, cat, horse, cow, and so forth.

Embodiments of the disclosure encompass methods of effecting gene therapy in an individual, comprising the step of delivering (such as by nanoparticle delivery, transfection, electroporation, hydrodynamic delivery, or a combination thereof) to the individual effective amounts of the first and second polynucleotides encompassed herein, said delivering step resulting in selective expansion of cells harboring the therapeutic polynucleotide. In specific cases, the second polynucleotide is delivered to the individual prior to, at the same time as, or subsequent to delivery of the first polynucleotide. In specific embodiments, following delivery of the first and second polynucleotides to the individual, expression of the essential gene product is disrupted at the second endogenous locus, and wherein the disruption is therapeutically treatable by delivering to the individual an effective amount of one or more nutritional or pharmacological agents to substitute for absence of the essential gene product. In some cases, the timing of the delivering of the one or more nutritional or pharmacological agents to the individual is dependent on a need of the individual. The one or more nutritional or pharmacological agents may be delivered to the individual to effect negative selective pressure on cells lacking the first polynucleotides. In specific cases, the one or more nutritional or pharmacological agents are delivered to the individual to effect positive selective pressure on cells harboring the polynucleotides.

Any individual that is a recipient of the system may have a medical condition related to the therapeutic polynucleotide, such as a liver medical condition. The individual may have a urea cycle disorder, branched chain amino acid disorder, amino acid disorder, or inborn error of metabolism with essential liver metabolism.

In specific embodiments, the essential gene product is fumarylacetoacetate hydrolase (Fah), fumarylacetoacetate hydrolase (FAH), dehydrodolichyl diphosphate synthase subunit (DHDDS), or 3-hydroxy-3-methylglutaryl Co-enzyme A reductase (HMGCR), UDP glucuronosyltransferase family 1 member A1 (UGT1A1), ormethylmalonyl coA mutase (MMUT). In particular embodiments, when the loss of Fah in cells transfected with the first and second polynucleotides is not needed in the individual, the individual is provided an effective amount of 2-(2-nitro-4-trifluoromethylbenzoyl)-1,3-cyclohexanedione (NTBC). In some cases, when the loss of Fah in cells transfected with the first and second polynucleotides is needed in the individual, the individual is provided an effective amount of a high protein diet.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Brief Summary, Detailed Description, Claims, and Brief Description of the Drawings.

### BRIEF DESCRIPTION OF THE DRAWINGS

For a more complete understanding of the present disclosure, reference is now made to the following descriptions taken in conjunction with the accompanying drawings.

FIG. 1. Targeted integration into the Apoa1 locus. An AAV vector (Repair Cassette) contains homology to the Apoa1 gene, and is inserted by homology-directed repair using CRISPR/Cas9 delivered by another AAV. The targeted locus can support expression of multiple transgenes downstream of Apoa1, through the use of 2A skipping peptides (shown) or IRES elements. In one embodiment, one transgene encodes an essential enzyme to be used for selection, the other cargo encodes a therapeutically relevant protein.

FIG. 2. Repair Drive as a novel approach to achieve permanent correction of monogenic liver diseases. In the first step, hepatocytes are metabolically poisoned through deletion of an essential enzyme. At the same time, the “antidote” is provided in the form of a promoterless integrating cassette. This AAV vector delivers the essential gene which is resistant to inhibition by CRISPR or shRNA. The therapeutically relevant protein is co-expressed from the same locus following genome editing. The correctly targeted cells are selectively expanded, where the degree of liver injury can be modulated by dietary or pharmacological means.

FIGS. 3A-3C. Study targeting a red fluorescent protein to the Apoa1 locus with AAV delivery. FIG. 3A) AAV vectors, experimental design, and timeline. FIG. 3B) In vivo editing efficiency by Sanger sequencing. FIG. 3C) Most common indel mutations introduced into the Apoa1 3′UTR determined by ICE. FIG. 3C discloses SEQ ID NOS 27-40, respectively, in order of appearance.

FIGS. 4A-4B. On-target integration at the Apoa1 locus in vivo. FIG. 4A) Diagram of the repair cassette used in the study in FIG. 3, showing the two major outcomes-NHEJ insertion of the whole vector, and correct HDR. FIG. 4B) PCR detection of integration events showing the presence of both NHEJ and HDR insertions in mice treated with the repair cassette and AAV-CRISPR.

FIGS. 5A-5B. Apoa1 targeting supports expression of a fluorescent reporter gene in fresh liver slices. FIG. 5A) Direct fluorescence for the mKate2 transgene shown in FIG. 3 above (red cells). FIG. 5B) Immunohistochemistry of paraffin sections showing correctly targeted hepatocytes (brown cells).

FIGS. 6A-6C. Human Factor IX can be expressed from the Apoa1 locus and secreted following AAV-CRISPR targeting. FIG. 6A) Vector and experimental design. FIG. 6B) Total ApoA1 levels are not adversely affected by editing, but 2A-tagged ApoA1 can be secreted. FIG. 6C) High levels of Factor IX at 6 and 12 weeks after AAV administration.

FIGS. 7A-7B. Successful expression and secretion of human ApoE with Apoa1 targeting. FIG. 7A) Experimental design for knocking in to the Apoa1 locus. FIG. 7B) Western blot for human ApoE in mouse plasma following AAV administration.

FIGS. 8A-8C. Selective expansion of gene-targeted hepatocytes using Fah as a selectable marker in the Fah KO mice. FIG. 8A) Targeting strategy to knock in the C-terminus of the LDLR gene into the native Ldlr locus, upstream of Fah and mKate2. FIG. 8B) Fah immunostaining on livers 12 weeks after AAV injection. Rare positive cells are present on 100% NTBC which are clonally expanded through NTBC cycling. FIG. 8C) PCR to detect the relative abundance of NHEJ versus HDR insertions. Selective expansion by NTBC cycling repopulates the liver with correctly targeted cells (HDR).

FIGS. 9A-9C. Dose response of AAV-CRISPR for deletion of endogenous Fah (i.e. the poison pill). FIG. 9A) Vector and experimental design. Mice are maintained on 100% NTBC so that Fah removal can be assessed without hepatocyte death and regeneration.

FIG. 9B) Western blot for Fah showing a dose-dependent reduction. FIG. 9C) Immunostaining for Fah+ hepatocytes 4 weeks after AAV injection.

FIGS. 10A-10C. Design and testing of AAV-shRNA to remove endogenous Fah. FIG. 10A) AAV vector expressing an shRNA to Fah as well as a GFP reporter gene. FIG. 10B) Initial screening of shRNA effectiveness in HEK293T cells. Note that twice as much Fah cDNA was transfected in lane 1, relative to shRNA groups on the right. FIG. 10C) Immunostaining showing effective Fah removal using AAV delivery of shRNA3 at one month after injection.

FIGS. 11A-11B. DHDDS as an essential gene that can be leveraged for expansion. FIG. 11A) Depicts a simplified diagram of the mevalonate pathway which produces cholesterol, dolichols, and other nonsterol isoprenoids (not shown). HMGCR is the rate-limiting enzyme, and DHDDS is a committed step to dolichol production. FIG. JIB) Dolichol is an essential metabolite required for glycosylation of proteins. Depletion of dolichol leads to ER stress and apoptosis. Dolichol can be depleted by inhibition, knockdown, or disruption of the DHDDS enzyme. Further selective pressure can be applied with dietary cholesterol, which suppresses HMGCR activity upstream, reducing the flux of isoprenoid substrates to DHDDS. Cells harboring an integrated DHDDS transgene will be resistant to cell death.

FIGS. 12A-12I: Selective expansion of ApoA1-targeted cells in adult mice using Dhdds as the essential gene. FIG. 12A) Diagram of AAV vectors used in the study: 1) ApoA1 gRNA AAV-CRISPR (5*10¹¹); 2) Dhdds gRNA AAV-CRISPR (1*10¹²); 3) Repair AAV (5*10¹¹). gRNAs and Staphylococcus Aureus Cas9 (SaCas9) are under the control of U6 and hepatocyte-specific HLP promoter, respectively. hDHDDS has been used as selectable marker. FIG. 12B) Timeline of the study: 8 weeks old C57BL/6J mice were injected with AAVs or saline (control) at time 0 and fed a chow or 1% cholesterol-enriched diet for 12 weeks. Blood was collected every two weeks for ALT measurement. Liver was harvested 12 weeks post-injection for evaluation of ApoA1-targeted cells. Experimental groups are indicated on the left (n=5). FIG. 12C) Body weight and FIG. 12D) ALT measurement over time. Purple line: control mice (chow); orange line: gRNAs-injected mice (chow); black line: gRNAs+Repair-injected mice (chow); green line: control mice (1% cholesterol); red: gRNAs-injected mice (1% cholesterol); blue line: gRNAs+Repair-injected mice (1% cholesterol). ***p<0.001 and *p<0.05: gRNAs (1% cholesterol) vs control (chow) and control (1% cholesterol) respectively at 4 and 5 weeks post-injection. ****p<0.0001 gRNAs (1% cholesterol) vs all the other groups at 4 weeks post-injection. FIGS. 12E, 12F) PCR for detecting the targeted integration at ApoA1 locus in livers from chow (FIG. 12E) and 1% cholesterol (FIG. 12F) diet fed mice. The HDR integration results in a band of 1024 bp, whereas the viral genome integration (including the ITRs) results in band of ˜2000 bp. “-”: no DNA; “no exp” (no expansion control): integration PCRs on livers targeted at the ApoA1 locus without using any selectable markers. FIG. 12G) Representative direct fluorescence (top) and immunohistochemistry (bottom) of mKate2-positive hepatocytes on livers from control mice (chow). Similarly, no positive staining was observed in gRNAs-injected (chow), control (1% cholesterol) and gRNAs-injected (1% cholesterol) mice. FIG. 12H) Representative direct fluorescence (top) and immunohistochemistry (bottom) of mKate2-positive hepatocytes on livers from gRNAs+Repair-injected mice (chow). FIG. 12I) Representative direct fluorescence (top) and immunohistochemistry (bottom) of mKate2-positive hepatocytes on livers from gRNAs+Repair-injected mice (1% cholesterol). Magnification and exposure time in fluorescent microscopy are 4× and 130 ms. Scale bar in IHC is 100 μM.

While various embodiments of the disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the disclosure described herein may be employed.

### DETAILED DESCRIPTION

### I. Definitions

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As defined herein, the terms “targets” or “target” or targeting” refer to the ability of a composition to be able to specifically bind (directly or indirectly) to a particular nucleic acid sequence. In specific embodiments, the composition itself comprises nucleic acid and the particular nucleic acid to which it binds is known. The composition may be desired for the purpose of targeting based on the known particular nucleic acid sequence. Examples of compositions that can target include guide RNAs or shRNAs or siRNAs.

As used herein, the term “co-expression” refers to the therapeutic polynucleotide and the essential gene product polynucleotide being expressed, at least initially, as the same nucleic acid molecule. Subsequent steps provide for separation of their respective gene products.

As defined herein, the terms “essential gene” or “essential gene product” refer to a gene or polypeptide produced from the gene without which a cell would die or have a growth disadvantage.

As defined herein, the term “nutritional or pharmacological agent” refers to exogenous substances, with respect to an individual, that are able to biologically compensate for loss of an essential gene product. The substances may or may not commonly or otherwise be known or utilized nutritionally or pharmacologically but nevertheless are able to nutritionally or pharmacologically substitute for loss of an essential gene product.

### II. General Embodiments

The present disclosure concerns systems, compositions, and methods related to gene therapy in an individual in need thereof. The gene therapy provides correction of at least one genomic locus in an individual that has at least one defective gene resulting in a medical condition directly or indirectly caused by the defective gene. The defective gene (which may be genomic or mitochondrial) may comprise a point mutation, duplication, inversion, copy number defect, or combination thereof. In particular embodiments, the defective gene is replaced with a wild-type copy of the gene, although in specific cases the replacement therapeutic gene has differences in sequence compared to the wild-type copy of the gene so long as those differences are not disease-causing and allow for production of functional activity of the respective gene product. In some cases, the therapeutic gene is inserted in place of the defective gene (i.e., at that locus), or instead is inserted at a safe harbor site, such as Apoa1.

### III. Systems

Systems and other compositions of the disclosure are utilized for effecting gene therapy in an individual. The system utilizes multiple polynucleotides having respective roles for therapeutically replacing a defective gene in vivo in a mammal. In particular embodiments, the system is configured such that cells in which a defective gene is replaced are able to expand in vivo in an environment under conditions that are deleterious for cells that lack an essential gene. Cells in the system that lack the therapeutic gene of the gene therapy die or eventually apoptose because of severe growth disadvantage, because they lack an essential gene to which the therapeutic gene is linked, such as transcriptionally linked, in at least some embodiments.

Embodiments of the disclosure include systems, comprising: (a) a first polynucleotide comprising an expression cassette, said expression cassette comprising a therapeutic polynucleotide linked to an essential gene product polynucleotide, wherein said cassette comprises one or more sequences capable of integrating at least part of the cassette at a first endogenous locus; and (b) a second polynucleotide comprising a targeting region that disrupts expression of the second endogenous locus, wherein said second endogenous locus encodes the essential gene product. In some cases, the second polynucleotide is not integrating at a locus. For example, the second polynucleotide may be an AAV vector expressing CRISPR/Cas9 to disrupt the second endogenous locus. Alternatively, the second polynucleotide is an siRNA or anti-sense oligonucleotide that may be repeatedly administered to knock down the essential gene at the second locus.

The therapeutic polynucleotide and the essential gene product polynucleotide may be linked by an element that allows for eventual production of separate polypeptides for the therapeutic gene product and the essential gene product, such as a 2A element or an IRES element. The therapeutic polynucleotide and the essential gene product polynucleotide may be configured in any suitable way, such as wherein in a 5′ to 3′ direction in the expression cassette, the therapeutic polynucleotide is 5′ or 3′ to the essential gene product polynucleotide.

In specific cases, the targeting region in the system comprises nucleic acid sequence that allows for targeting at a specific nucleic acid sequence in a DNA, such as genomic DNA of an individual in need of the therapeutic gene. The targeting region may comprise sequence that expresses sequence that is complementary to at least part of the second endogenous locus. Examples of the targeting region include guide RNA sequence for a CRISPR/Cas9 system, ZNF or other designer nucleases, shRNA, or siRNA.

In particular embodiments for the system, the first polynucleotide and/or the second polynucleotide are present in an integrating vector, such as an adeno-associated viral vector, lentiviral vector, or retroviral vector. In other cases, the first polynucleotide and/or the second polynucleotide are present in a non-integrating vector, such as a plasmid or adenoviral vector. The system is configured such that the integration at the first endogenous locus may be targeted or random integration. In examples of targeted integration, the first endogenous locus may be selected based on the ability of the endogenous locus to provide robust expression of the integrated expression construct, and in such cases the expression construct may or may not comprise regulatory sequence(s), such as a promoter, to effect expression.

In particular embodiments, the system is configured such that when there is disruption of expression at the second endogenous locus that encodes the essential gene product, the loss of the essential gene product may be substitutable by presence of one or more nutritional or pharmacological agents in the individual, including in the transfected cells. That is, the one or more nutritional or pharmacological agents mask the loss of the essential gene product by providing activity that circumvents absence of the essential gene product itself (such as a downstream product of the same pathway). Thus, for the individual harboring cells of the system, disruption of expression at the second endogenous locus that encodes the essential gene product is therapeutically treatable by one or more nutritional or pharmacological agents to substitute for absence of the essential gene product. In some cases, loss of an essential metabolic or gene function may be rescued by supplementing the essential metabolite. In other cases, accumulation of a toxic product is prevented by blocking the pathway upstream (i.e., nitisinone).

To prevent loss of the essential gene product polynucleotide of the system when production of the endogenous essential gene product is being disrupted, the essential gene product polynucleotide may be configured to be resistant to disruption of expression by the targeting region, such as with sequence variants (for example, using different codons). In some embodiments, the system could allow for targeting of noncoding sequence at the second endogenous locus (for example, endogenous noncoding genes such as microRNA or long noncoding RNA could be the essential gene that is removed).

The system may be utilized for any therapeutic purpose for which gene therapy is efficacious. The system may be utilized for any tissue of a mammal. In specific cases, the system is therapeutic for a liver medical condition. In such cases, the first endogenous locus may be ApoA1 or albumin, for example, and/or the essential gene product may be fumarylacetoacetate hydrolase or dehydrodolichol diphosphate synthase subunit, for example.

With respect to the system elements that allow for linkage of expression between the therapeutic polynucleotide and the essential gene product polynucleotide, any element may be used to ensure that the presence of the therapeutic polynucleotide requires the presence of the essential gene product polynucleotide. An exemplary element is a site that encodes a self-cleaving peptide, such as a 2A peptide cleavage sequence. Other cleavage sites include furin cleavage site or a Tobacco Etch Virus (TEV) cleavage site. In other cases, they may be linked by one or more elements that provide for distinct translation of the separate polypeptides (such as IRES sequences). In embodiments wherein self-cleaving 2A peptides are utilized, the 2A peptides may be 18-22 amino-acid (aa)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. The mechanism of 2A-mediated “self-cleavage” was discovered to be ribosome skipping the formation of a glycyl-prolyl peptide bond at the C-terminus of the 2A. A highly conserved sequence GDVEXNPGP is shared by different 2As at the C-terminus, and is useful for the creation of steric hindrance and ribosome skipping. Successful skipping and recommencement of translation results in two “cleaved” proteins. Examples of 2A sequences are as follows:

T2A:
(SEQ ID NO: 1)
(GSG)EGRGSLLTCGDVEENPGP
P2A:
(SEQ ID NO: 2)
(GSG)ATNFSLLKQAGDVEENPGP
E2A:
(SEQ ID NO: 3)
(GSG)QCTNYALLKLAGDVESNPGP
F2A:
(SEQ ID NO: 4)
(GSG)VKQTLNFDLLKLAGDVESNPGP

### IV. Methods

Embodiments of the disclosure provide methods of effecting gene therapy in an individual. The gene therapy may be for any medical condition in the individual and may or may not be associated with defects in a particular tissue of the individual. In specific embodiments, the tissue is the liver and the methods are well-suited to the liver given its capacity for regeneration. In some embodiments, the tissue is the brain, muscle, kidney, bone, spleen, gall bladder, lungs, bladder, kidneys, heart, stomach, intestines, and so forth.

Methods of the disclosure allow for gene therapy in an individual by imparting selective pressure on cells that have the replaced, therapeutic gene. Such selective pressure is effective because the presence of the therapeutic gene is linked to the presence of a marker that is an essential gene. Those cells that have the therapeutic gene linked to the essential gene are not subjected to death for lacking the essential gene product. In particular, those cells that have the therapeutic gene linked to the essential gene are safe from death and able to expand when the tissue is exposed to one or more agents that are lethal to the cells in the absence of the essential gene product.

Methods of the disclosure utilize the system encompassed herein: (a) a first polynucleotide comprising an expression cassette, said expression cassette comprising a therapeutic polynucleotide linked to an essential gene product polynucleotide, wherein said cassette comprises one or more sequences capable of integrating at least part of the cassette at a first endogenous locus; and (b) a second polynucleotide comprising a targeting region that disrupts expression of the second endogenous locus or activity of a gene product produced therefrom, wherein said second endogenous locus encodes the essential gene product. In specific embodiments, there is no integration at the second endogenous locus; instead, the locus may be knocked out by one of a variety of methods.

Embodiments of the disclosure provide for methods of effecting gene therapy in an individual, comprising the step of delivering to the individual effective amounts of the first and second polynucleotides of the system. Following delivery of the first and second polynucleotides to the individual, expression of the essential gene product becomes disrupted at the second endogenous locus. Cells in the tissue exposed to the first polynucleotide in the system include those that were also transfected with the second polynucleotides and those that were not transfected with the second polynucleotide. Those cells that were transfected with the second polynucleotide but lack integration of the essential gene product will ultimately die, particularly when there is selective pressure applied. Such selective pressure can be increased upon exposure to one or more nutritional or pharmacological agents that require presence of the essential gene product in the cells to survive.

In some embodiments, it is undesirable to impart selective pressure on the system-transfected cells. Examples include when the selective pressure becomes harmful to the individual. In specific embodiments, the disruption of expression of the endogenous essential gene product is therapeutically treatable by delivering to the individual an effective amount of one or more nutritional or pharmacological agents to substitute for absence of the essential gene product. This is a controllable aspect to the system, and the timing of the delivering of the one or more nutritional or pharmacological agents to the individual may be dependent on a need of the individual. In some cases, the one or more nutritional or pharmacological agents are delivered to the individual to effect negative selective pressure on cells lacking the first and second polynucleotides. In other cases, the one or more nutritional or pharmacological agents are delivered to the individual to effect positive selective pressure on cells harboring the first and second polynucleotides.

In some embodiments, there are methods of treating an individual for a medical condition by subjecting the individual to the system of the disclosure. In specific embodiments, the individual has a medical condition related to the therapeutic polynucleotide, such that correction of the corresponding endogenous gene of the therapeutic polynucleotide treats at least one symptom of the medical condition. In specific cases, the individual has a liver medical condition. In particular aspects, when the individual has a liver medical condition, the essential gene product is fumarylacetoacetate hydrolase (Fah). In a modular attribute of the system, when the loss of Fah in cells transfected with the first and second polynucleotides is not needed in the individual with the liver medical condition, the individual is provided an effective amount of 2-(2-nitro-4-trifluoromethylbenzoyl)-1,3-cyclohexanedione (NTBC). On the contrary, when the loss of Fah in cells transfected with the first and second polynucleotides is needed in the individual with the liver medical condition, the individual is provided an effective amount of a high protein diet.

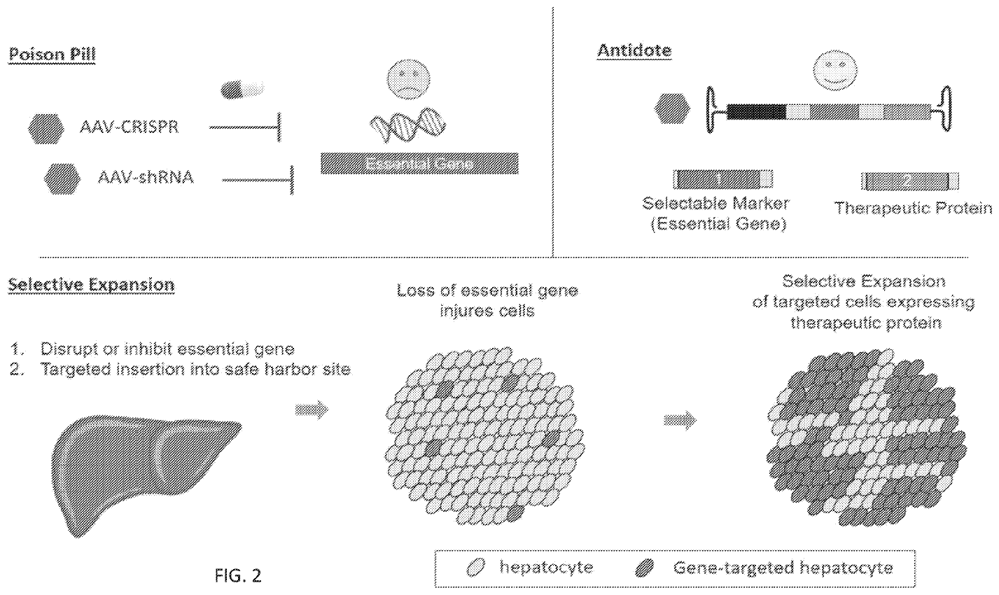

In embodiments wherein the individual has a liver medical condition, selective expansion of gene-targeted hepatocytes can occur in certain metabolic liver diseases where there is a survival advantage (14-18). In these situations, cells integrating a functional copy of the defective gene will gradually repopulate the liver. Although this only occurs naturally in a subset of liver diseases, this survival embodiment may be utilized to improve the efficiency of gene therapies requiring targeted integration. The present disclosure utilizes deletion of an essential gene from the liver, while simultaneously replacing it in gene-targeted hepatocytes. In this way, cells harboring a permanent copy of a therapeutic transgene can be selectively expanded. A feature of the approach is that the gene-targeted cells express the endogenous gene that was used for selection, ultimately restoring normal liver physiology (i.e., another metabolic disease is not generated in the process). One embodiment of this system is shown in FIG. 2.

In particular embodiments, the systems, methods, and compositions are related to medical conditions associated with any kind of tissues or cells. In particular embodiments, the individual has a liver medical condition, such as an infection (such as any kind of hepatitis including A, B, or C); Autoimmune hepatitis; Primary biliary cirrhosis; Primary sclerosing cholangitis; Hemochromatosis; Hyperoxaluria and oxalosis; Wilson's disease; Alpha-1 antitrypsin deficiency; Liver cancer; Bile duct cancer; Liver adenoma; Chronic alcohol abuse; Fat accumulating in the liver (nonalcoholic fatty liver disease), inborn errors of metabolism because of liver-expressed genes such as, but not limited to, urea cycle disorders and branched-chain amino acid disorders, or a combination thereof.

In examples of the present disclosure, one can determine if Apoa1 targeting can promote durable expression of therapeutic transgenes. In specific embodiments, the Apoa1 locus is an example of a useful site for targeted insertion of therapeutic transgenes in the liver. To characterize this, AAV vectors are used to deliver CRISPR/Cas9 and a donor template with homology to the 3′ untranslated region of Apoa1. Successful integration allows for expression of a therapeutic gene from the same mRNA, using either 2A or IRES elements (for example). The efficiency of Apoa1 targeting with a fluorescent reporter may be used to optimize guide RNAs and repair template design. Unbiased sequencing may be used to assess the risk of off-target cutting and insertional mutagenesis, and to fully characterize on-target integrations. One can determine if expression from Apoa1 can support high level expression of the secreted proteins factor IX (FIX) and APOE, as examples. Phenotypic correction of hyperlipidemia and atherosclerosis may be determined through targeted insertion of human APOE into livers of Apoe KO mice.

In particular embodiments, there is a flexible system for selective expansion of gene-targeted cells of any kind, including at least hepatocytes, for example. Correction of many liver disorders by any means will require efficient genome editing in a large proportion of hepatocytes. The rate of targeted insertions via HDR is expected to be low, limiting this method to diseases with a low threshold of correction. However, in the present disclosure, a targeted integration approach is leveraged to promote selective expansion of gene-targeted hepatocytes. In specific embodiments, to accomplish this an essential gene (Fah) is deleted in the majority of the liver with AAV-CRISPR, as one example. At substantially the same time, cells with targeted insertions of the therapeutic transgene can also restore expression of the essential gene. Over time, the edited cells repopulate the liver, enabling more robust and permanent transgene expression. The selection pressure can be titrated in both directions. A drug that blocks the catabolic pathway upstream and prevents accumulation of toxic catabolites (2-[2-nitro-4-(trifluoromethyl)benzoyl] cyclohexane-1,3-dione; also known as nitisinone; NTBC) will preserve liver function. Selection pressure can be increased by withdrawing the drug and/or feeding a high protein diet. In some cases, selective expanstion may be assessed by immunostaining, deep sequencing, and/or restoration of FIX and/or APOE levels (as examples only).

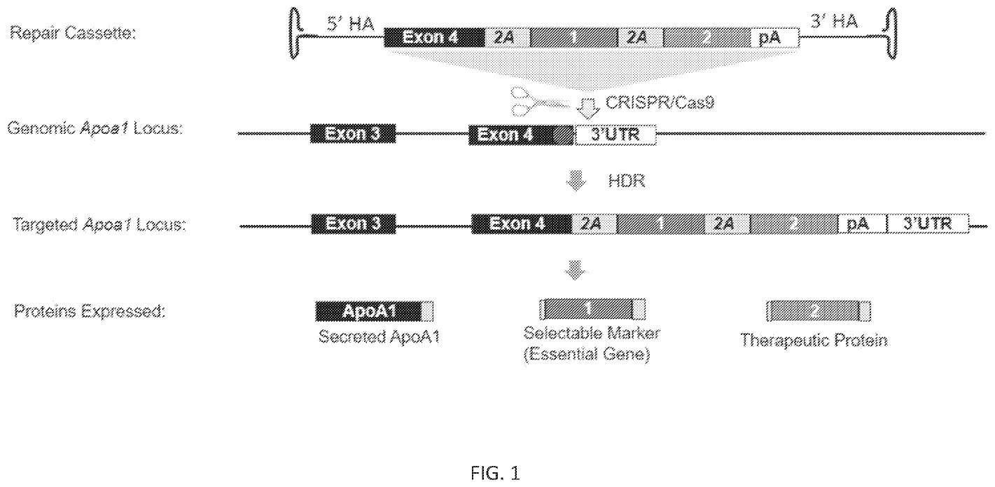

In specific embodiments of the disclosure, targeted integration of the first and second polynucleotides is utilized, because heritable changes in hepatocytes are passed on to daughter cells. Achieving this requires the identification of safe harbor sites that can support expression of therapeutic transgenes without adverse consequences. There has already been considerable work in targeting the Albumin (Alb) locus with AAV donors for homologous recombination. These strategies can achieve therapeutically relevant levels of certain transgenes (i.e. Factor IX, Factor VIII, etc.), despite the low inefficiency of targeting (˜1%). Upcoming clinical trials should provide valuable information about how this approach compares to conventional gene therapy (NCT02695160, NCT02702115, NCT03041324). However, recent studies have identified the Albumin gene as frequently mutated in hepatocellular carcinoma biopsies (11-13). The present disclosure characterizes the Apoa1 locus as a safe harbor site as an additional option. The general concept of Apoa1 targeting with AAV and CRISPR is shown in FIG. 1.

In particular cases, AAV vectors can deliver a CRISPR/Cas9 to the liver, and edit genes with high efficiency. CRISPR/Cas9 cutting greatly increases the efficiency of homology-directed repair (HDR), and can also be used for homology independent integrations (HITI). In this disclosure, the Apoa1 gene is demonstrated to be an effective safe harbor site for transgene insertion with AAV. Apolipoprotein A1 (Apoa1) is the major structural component of high density lipoproteins and one of the most abundant proteins in plasma (˜1 mg/ml). AAV is used deliver CRISPR/Cas9 to open the Apoa1 locus and insert transgenes, where they are driven by the highly active Apoa1 promoter. This system is characterized by expressing fluorescent reporters, as well as examples of therapeutic transgenes—Factor IX (FIX) and Apolipoprotein E (ApoE). Another embodiment allows for improvement of the degree of correction by promoting selective expansion of the gene-targeted cells, greatly broadening the range of liver diseases that can be treated. The strategy allows for deletion of an essential enzyme (as one example, Fah) in order to metabolically poison hepatocytes. At the same time, the essential gene is replaced in a subset of cells through targeted integration. The degree of liver injury and selective pressure can be increased (high protein diet) or decreased (NTBC) as needed. Over time, cells expressing the therapeutic transgene proliferate and repopulate the liver. Importantly, the gene-corrected hepatocytes retain expression of the essential gene, preserving normal liver metabolism and physiology upon expansion. In a specific embodiment, precise targeting of the Apoa1 locus allows for durable expression of therapeutic transgenes, and these gene-corrected cells can be expanded using an essential gene for selection.

### EXAMPLES

The following examples are included to demonstrate particular embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Apoa1 Targeting and Promotion of Durable Expression of Therapeutic Transgenes

Limitations of Liver-Directed AAV Gene Therapy.

Great progress has been made in liver-directed gene therapy with AAV vectors, including Phase II trials for Hemophilia B and Hemophilia A. However, the long-term durability of AAV gene therapy remains to be determined. Recombinant AAV vectors are non-integrating, and circularize in the nucleus to form stable episomes (7). These episomes are expected to be lost through cell death and division. It has been estimated that the typical lifespan of a hepatocyte is between 200 and 400 days (8,9), so it is a reasonable prediction that conventional AAV gene therapy will not provide lifelong correction. Targeted integration into a safe harbor locus would allow for more permanent expression, as the changes to the genome would be heritable to daughter cells. In the context of liver gene therapy, the Albumin (Alb) locus has been used for ‘promoterless targeting,’ where AAV repair templates integrate the therapeutic transgene. However, recent data have defined Albumin as one of the most significantly mutated genes in human Hepatocellular Carcinoma (12,13) with mutations in this gene observed in 13% of tumors (11). Therefore, there is a compelling need to identify other viable safe-harbor sites for liver-directed genome editing with AAV vectors.

In specific embodiments of the disclosure, the following are examples of criteria for a safe harbor locus, and one or more may be applicable to the locus:
1) The integration site has accessible chromatin that is amenable to precise gene insertion, for example through homology directed repair (HDR) or homology independent targeted integration (HITI), such as with CRISPR/Cas9.
2) The safe harbor locus drives high-level expression of therapeutic transgenes in the desired tissue or organ, such as the liver.
3) The targeting event does not compromise the function of important neighboring genes.
4) The expression cassette may be “promoterless” in order to maximize transgene cargo capacity, but also to minimize the risks of off-target integrations that could be deleterious, such as cause cancer.

One embodiment of a safe harbor locus is Apolipoprotein A1 (Apo A1). Apo A1 is a secreted protein that is the main structural component of high density lipoproteins (HDL). It is present in plasma at concentrations of 1 mg/mL, making it one of the most abundant secreted proteins produced by the liver. The relatively small size of the Apoa1 gene, well studied biology, and accessibility of chromatin at this locus, make it a useful candidate for targeted insertion. In a specific embodiment, one tests whether the Apoa1 gene is a suitable docking site for targeted integration using AAV-CRISPR. This may be determined using fluorescent reporters, targeted and unbiased deep sequencing, and/or expression of therapeutically relevant transgenes. The durability of expression may be assessed through rescue of hyperlipidemia and atherosclerosis in the apolipoprotein E knockout (Apoe KO) mice with human APOE, for example.

In particular embodiments, the Apoa1 locus is a useful safe harbor site for targeted integration of AAV transgenes, and provides sustained levels of therapeutic protein expression in the liver.

In certain embodiments, one may utilize albumin or other highly expressed liver genes as an alternative to Apo A1 as a safe harbor gene. This concept of “promoterless targeting” was introduced by Barzel et al. (43), and involves the use of a 2A skipping peptide to express transgenes from the C-terminus of the Albumin mRNA. Although the actual targeting efficiency is low (˜0.5% of hepatocytes), this strategy works well for secreted proteins because albumin is so highly expressed in the liver. AAV-based targeting of albumin, termed “GeneRide” has recently been used to correct Alpha 1 anti-trypsin deficiency (44) as well as Crigler-Najjar syndrome (45) in mice. Zinc Finger Nucleases (ZFN) can improve the efficiency targeting, supporting robust expression of Factor VIII, Factor IX, and several lysosomal storage disorder enzymes (46). Hunter's syndrome (47) and Hurler's syndrome (48) have both been corrected in mouse models through liver-directed targeting of Albumin using Zinc Finger Nucleases. This work has enabled Phase I clinical trials to treat Hemophilia B (NCT02695160), Mucopolysaccharidosis I (MPS I) (NCT02702115), and Mucopolysaccharidosis II (MPS II) (NCT03041324). However, Albumin remains the only successful example to date of a common safe harbor site for liver-directed gene therapy.

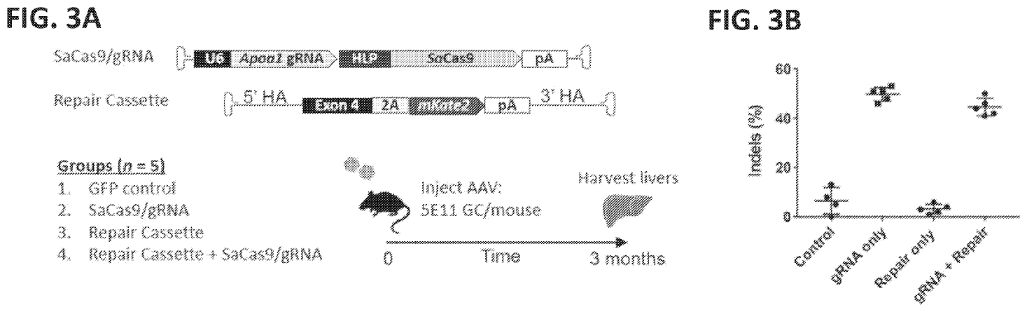

Targeting the ApoA1 locus with AAV and CRISPR/Cas9. To characterize the feasibility of targeting the Apoa1 locus with CRISPR/Cas9, a gRNA to the 3′ untranslated region (3′UTR) of Apoa1, downstream of the stop codon, was designed. An AAV8 vector was built expressing this gRNA, along with Staphylococcus aureus Cas9 (SaCas9) driven by a liver specific promoter (SaCas9/gRNA). In addition, a promoterless AAV8 vector was constructed to enable insertion of a far-red fluorescent protein reporter (mKate2) into the Apoa1 locus, using a P2A skipping peptide. This “repair cassette” also has homology arms to the Apoa1 gene to facilitate integration through homology directed repair (HDR). Mice were injected with AAV vectors and followed for three months (FIG. 3A). Sanger sequencing and analysis of indels by decomposition showed a high efficiency of indel formation in the Apoa1 3′UTR in the livers of mice receiving SaCas9/gRNA and the SaCas9/gRNA and repair cassette together (FIGS. 3B, 3C). Sequences from FIG. 3C are as follows:

| | | | |
|---|---|---|---|
| GAAAGGTTTATTG | SEQ ID | TGCGGGGGTGGGGAGTGGAAGCGG | SEQ ID |
| TAAGAAAGCCAA | NO: 5 | GCACCTCACTGGGCAGTCAGAGTCT | NO: 22 |
| | | C | |
| GAAAGGTTTATTG | SEQ ID | NTGCGGGGGTGGGGAGTGGAAGCG | SEQ ID |
| TAAGAAAGCCAA | NO: 5 | GGCACCTCACTGGGCAGTCAGAGT | NO: 23 |
| | | CT | |
| GAAAGGTTTATTG | SEQ ID | GCGGGGGTGGGGAGTGGAAGCGGG | SEQ ID |
| TAAGAAAGCCAA | NO: 5 | CACCTCACTGGGCAGTCAGAGTCTC | NO: 24 |
| GAAAGGTTTATTG | SEQ ID | CGGGGGTGGGGAGTGGAAGCGGGC | SEQ ID |
| TAAGAAAGCCAA | NO: 5 | ACCTCACTGGGCAGTCAGAGTCTC | NO: 25 |
| GAAAGGTTTATTG | SEQ ID | NNTGCGGGGGTGGGGAGTGGAAGC | SEQ ID |
| TAAGAAAGCCAA | NO: 5 | GGGCACCTCACTGGGCAGTCAGAG | NO: 14 |
| | | TC | |
| GAAAGGTTTATTG | SEQ ID | GTGGAAGCGGGCACCTCACTGGGC | SEQ ID |
| | NO: 6 | AGTCAGAGTC | NO: 15 |
| GAAAGGTTTATTG | SEQ ID | AGTGGAAGCGGGCACCTCACTGGG | SEQ ID |
| TAA | NO: 7 | CAGTCAGAGTCTC | NO: 16 |
| GAAAGGTTTATTG | SEQ ID | TGGAAGCGGGCACCTCACTGGGCA | SEQ ID |
| TAA | NO: 7 | GTCAGAGTCTC | NO: 17 |
| GAAAGGTTTA | SEQ ID | TGGAAGCGGGCACCTCACTGGGCA | SEQ ID |
| | NO: 8 | GTCAGAGTCTC | NO: 18 |
| GAAAGGTTTATTG | SEQ ID | GAGTGGAAGCGGGCACCTCACTGG | SEQ ID |
| AAG | NO: 9 | GCAGTCAGAGTCTC | NO: 19 |
| GAAAGGTTTATTG | SEQ ID | TGCGGGGGTGGGGAGTGGAAGCGG | SEQ ID |
| TAAGAAA | NO: 10 | GCACCTCACTGGGCAGTCAGAGTCT | NO: 22 |
| | | C | |
| GAAAGGTTTATTG | SEQ ID | GGGGGTGGGGAGTGGAAGCGGGCA | SEQ ID |
| TAAGAAAGCCAA | NO: 11 | CCTCACTGGGCAGTCAGAGTCTC | NO: 20 |
| GAAAGGTTTATTG | SEQ ID | TGCGGGGGTGGGGAGTGGAAGCGG | SEQ ID |
| TAAGAAAGCCA | NO: 12 | GCACCTCACTGGGCAGTCAGAGTCT | NO: 22 |
| | | C | |
| GAAAGGTTTATTG | SEQ ID | AGTGGAAGCGGGCACCTCACTGGG | SEQ ID |
| TAAG | NO: 13 | CAGTCAGAGTCTC | NO: 21 |

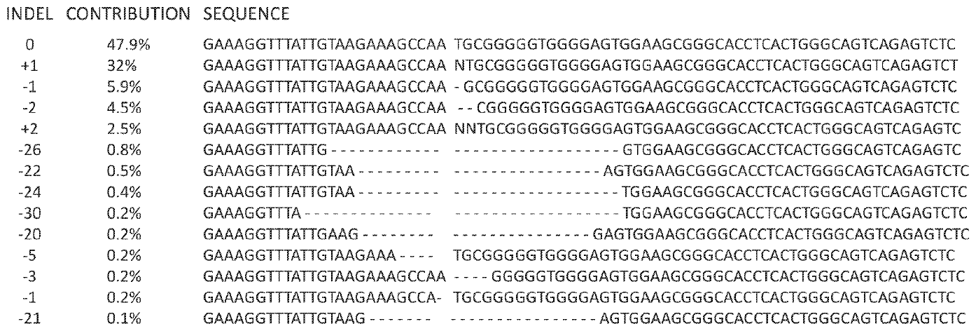

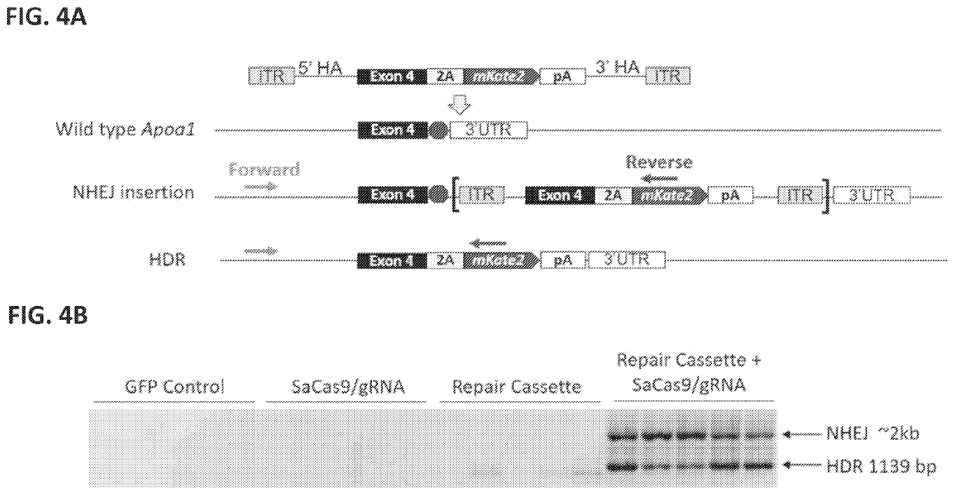

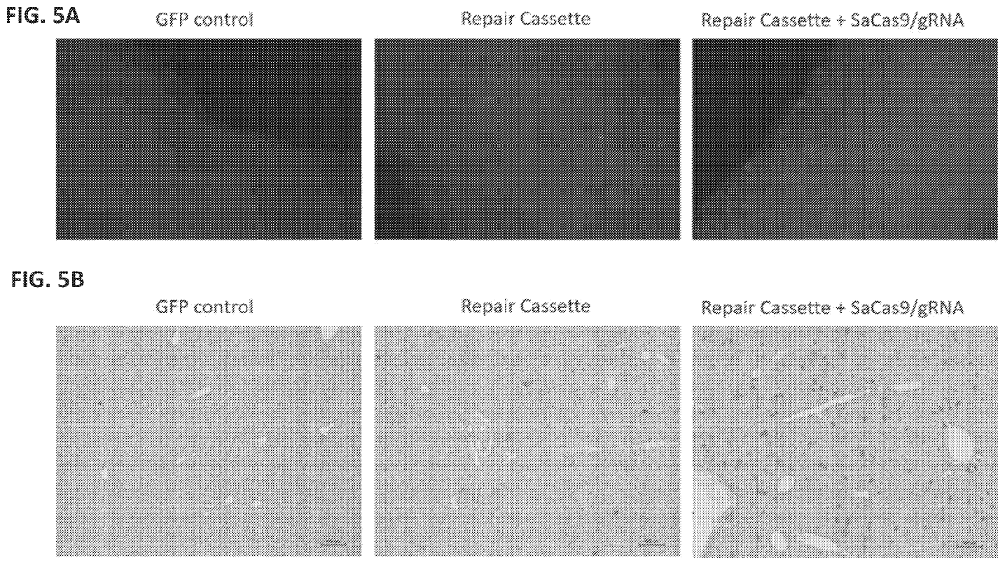

To identify on-target integrations, PCR was performed with a primer flanking the cut site in Apoa1, and another within the AAV repair template (FIG. 4A). The two bands were extracted, cloned, and sequenced. The top band represents insertion of the entire AAV repair template including the ITRs, while the bottom band is precisely repaired by HDR (FIG. 4B). Three months after injection, mKate2+ cells are visible at low frequency in livers receiving the repair template alone (FIG. 5A). AAV-CRISPR cutting of the target site dramatically increased the frequency of mKate2+ cells. This was also confirmed by immunohistochemistry staining for a FLAG tag on mKate2 (FIG. 5B).

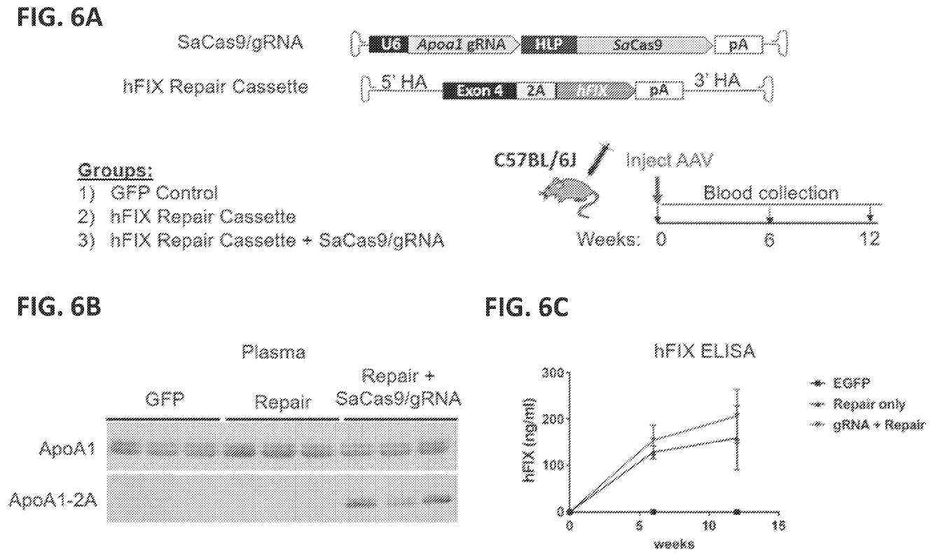

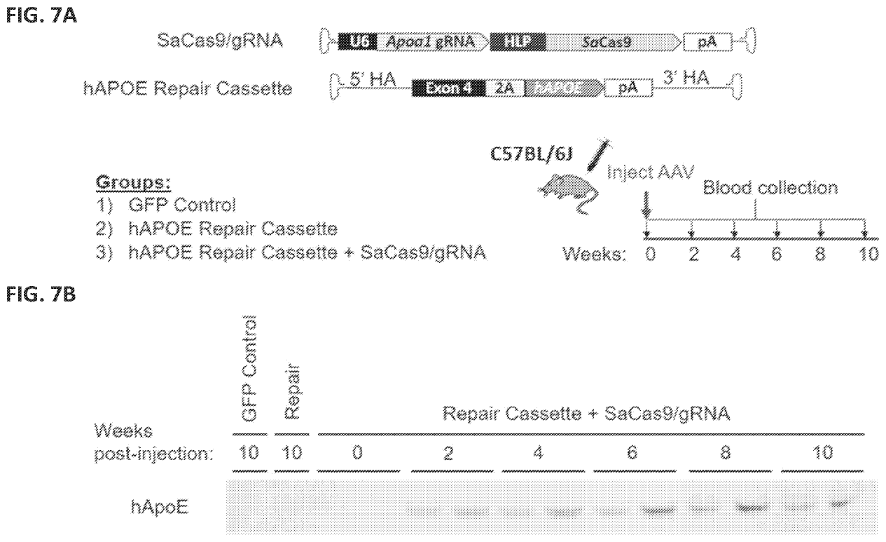

Expression of secreted transgenes from the Apoa1 locus. To characterize whether the Apoa1 gene modification could support expression of secreted proteins, a new repair template encoding Factor IX (FIX) was constructed. Mice were injected with either 1) a GFP control vector, 2) the FIX repair cassette, or 3) the FIX repair cassette plus SaCas9/gRNA (FIG. 6A). Western blotting of plasma showed that the total levels of Apo A1 in these mice were not adversely affected by gene targeting, and that a 2A-tagged version of Apo A1 is present in plasma, a useful readout of targeting efficiency (FIG. 6B). In addition, human FIX was readily detected in plasma at 6 and 12 weeks after AAV administration (FIG. 6C). Similar results were obtained in an experiment targeting the human APOE transgene to the Apoa1 locus of Apoe KO mice. In this case, human Apo E could be detected in plasma from at least 2-10 weeks after AAV injection by western blotting (FIGS. 7A, 7B).

Experimental Design.

Guide RNA design and testing. A gRNA was already identified that can cut the Apoa1 3′UTR. To find the most efficient possible gRNA, one can survey all possible designs within 500 bp downstream of the stop codon. These gRNA are cloned into a AAV-CRISPR plasmid vector (24), and tested using a split-luciferase system through transient transfection of HEK293T cells. In this assay, the luciferase coding sequence is interrupted by the gRNA target site, which is flanked by direct repeats (49). In a subset of repair events, the luciferase gene is restored by repair through single-strand annealing. This assay is used to identify the most efficient self-targeting gRNA for SaCas9(27), and one can use it as a quantitative readout of cutting efficiency. Firefly luciferase activity (gRNA activity) may be normalized to Renilla luciferase (transfection efficiency) for a minimum of 5 replicate wells per assay. Data is analyzed by one-way ANOVA followed by Tukey's posttest, with significance assigned at p<0.05. Expected Results—If there is identification of more efficient gRNA than the existing sequence, it can be used instead for in vivo studies.

Vector design and construction. AAV plasmids are generated using standard molecular biology approaches. An AAV-CRISPR vector to be used has been published (27), and expresses SaCas9 under the liver-specific HLP promoter of McIntosh et al. (50). The AAV repair templates may contain the final coding exon of Apoa1, fused to P2A skipping peptide and an mKate2 fluorescent reporter, followed by a small synthetic poly A signal. Surrounding these features, intronic and intragenic homology arms of 500 bp each to the Apoa1 locus are included. In addition, an identical repair vector is constructed that replaces the P2A skipping peptide with an IRES element. AAV vectors based on serotype 8 are produced by the triple transfection method of Xiao Xiao et al. (51) and purified by CsCl density gradient centrifugation (22).

Comparison of 2A and IRES elements for bicistronic expression. Data shows that one can perform targeted integration at the Apoa1 locus. This experiment expresses mKate2 from the Apoa1 transcript using a P2A skipping peptide. Next one can compare this approach to bicistronic expression with an IRES element. IRES elements have the advantage of leaving no novel amino acids on either protein, but are larger in size, and can result in lower levels of overall expression relative to 2A. To compare the relative efficiency of these approaches, one can inject C57BL6/J mice with AAV8 vectors at a dose of 5E11 GC per animal. These studies will require n=8 animals per group. All animal experiments may be performed in both male and female mice, to be analyzed separately. An example of groups are as follows: 1) Saline injected (negative control), 2) SaCas9/gRNA alone, 3) 2A-mKate2 repair alone, 4) 2A-mKate2 repair+SaCas9/gRNA, 5) IRES-mKate2 repair alone, 6) IRES-mKate2 repair+SaCas9/gRNA. Mice are followed for one month before sacrifice and tissue harvest. The percentage of mKate2+ cells in frozen liver sections are counted in a blinded fashion. The absolute level of mKate2 expression are compared across groups by western blotting for the FLAG epitope tag on the fluorescent protein.

In specific embodiments, there is no fluorescence or FLAG staining detected in the mice injected with saline or SaCas9/gRNA alone (groups 1 and 2). In specific cases, mice injected with each repair template alone (groups 3 and 5) have rare positive cells, in the range of 0.5-1.0% per liver. In specific cases, mice with the repair templates+SaCas9/gRNA have markedly more fluorescent cells, for example in the range of 5-10%. In a specific embodiment, there is a similar proportion of mKate2+ cells, with both the IRES and 2A vectors. The 2A-mKate2 reporter gives higher expression of mKate2+ per cell relative to the IRES construct, in particular embodiments.

Quantitation of on- and off-target cutting. The risk of off-target mutagenesis is a consideration with any genome editing approach. To determine the frequency and specificity of cutting with AAV-CRISPR, one can perform a targeted deep sequencing livers from the mice. Potential off-target sites for the gRNA targeting Apoa1 may be bioinformatically identified using the COSMID algorithm (https://crispr.bme.gatech.edu/)(33). The twenty most-likely off-target sites may be queried by targeted deep sequencing as published previously (24-27). Mice injected with saline may serve as the baseline to rule out PCR or sequencing error. Using this approach there is high sensitivity for off-target events, and can reliably detect mutagenesis even in the range of 0.2-0.5% for most sites. In specific embodiments, there is achievement of high rates of on-target mutagenesis at the Apoa1 locus. Given the restrictive Protospacer Adjacent Motif for SaCas9 (NNGRRT), in specific embodiments there is minimal off-target mutagenesis.

Identification of vector genome insertion sites. Recombinant AAV vectors are largely non-integrating in the absence of the Rep protein. However, with improved PCR and sequencing technologies it is becoming increasingly apparent that these vectors can integrate, albeit generally randomly and with low frequency (52). Nonetheless, there are examples where insertional mutagenesis with AAV can be problematic, including promoting liver cancer in mice injected as neonates, through integration into the Rian locus (53). Additionally, a recent study found wild type AAV2 integrations in a number of human hepatocellular carcinoma biopsies, which included several genes implicated in tumorigenesis (54). Lastly, the inventors (24,27,55) and others (56,57) have observed insertion of AAV vectors at CRISPR/Cas9 generated cut sites, which create artificial hotspots for integration. Therefore, an unbiased survey of AAV insertional mutagenesis may be performed. To accomplish this, one can perform ligation-mediated PCR. Genomic DNA may be sheared to an average size of 400-600 base pairs. Next, a double stranded adaptor oligo is ligated onto all blunt ends to provide a handle for PCR amplification. A primer specific to the inverted terminal repeats (ITRs) of the AAV genome is used together with a primer to the adaptor to amplify regions of AAV integration. The resulting PCR products may be barcoded and subjected to deep sequencing, for example. In specific embodiments, the analysis pipeline may first identify short regions of sequence unique to the AAV ITRs, and then align the adjacent sequences to the mouse genome may be determined. One can also use a variation of this approach to quantify and characterize on-target integrations. In this case, the gene-specific primer binds to the Apoa1 locus flanking the cut site. The reads are aligned to the AAV genome to determine the percentage of products arising from AAV insertion (ITR's present) versus HDR. Unbiased genomic sequencing is known in the art (29,58-61).

In specific embodiments, there is identification of AAV integrations at the on-target site in Apoa1 using the gene-specific primer that binds to the ITR. In specific embodiments, AAV insertions happens at off-target sites subject to CRISPR/Cas9 cutting. Additionally, there may be other places in the genome where the AAV can integrate, although these should be rare events. In particular embodiments, there is a high percentage of AAV vectors correctly integrated through HDR.

Durability of expression of secreted proteins—Targeted integration into a highly expressed locus in the liver is useful to express secreted proteins of therapeutic relevance. To characterize the capacity of Apoa1 targeting to support sustained expression, one can use AAV8 repair templates encoding either human Factor IX (FIX) or Apolipoprotein E (Apo E). C57BL6/J mice are injected with AAV vectors at 8 weeks of age at a dose of 5E11 GC per animal. The groups (n=8) may be as follows: 1) Saline injected (negative control), 2) SaCas9/gRNA alone, 3) 2A-FIX Repair alone, 4) 2A-FIX repair+SaCas9/gRNA. The same group design may also be used for human APOE in place of FIX, in some cases. One can also substitute IRES for 2A if needed, pending the results of the previous experiments with the mKate2 reporter. Plasma may be obtained before injection, and then again at 2, 4, 6, 8, 12, and 24 weeks afterwards. At 6 months post-injection, mice may be sacrificed to harvest liver and other peripheral tissues. Human Factor IX levels are measured in the plasma using an ELISA Kit. Human Apo E protein levels are measured by western blotting as have been performed previously (21). In specific embodiment, one can detect both FIX and APOE in the plasma. Levels of these proteins are detectable at 2 weeks after AAV administration, and ramp up to a steady state by 6 weeks that is maintained out to 6 months post-delivery, in specific cases. In some cases, there may be significantly higher levels of FIX and APOE in mice receiving the SaCas9/gRNA and repair cassette, relative to the repair cassette alone.

Correction of hyperlipidemia and atherosclerosis. Apo E is a secreted apolipoprotein that helps in the transport of cholesterol and triglycerides in the bloodstream. Apo E is found on chylomicrons, very low-density lipoprotein (VLDL), intermediate density lipoprotein (IDL), and high density lipoprotein particles. This protein is the high affinity ligand for the low density lipoprotein receptor (LDLR), which is responsible for the clearance of ApoB-lipoproteins by the liver. The APOE gene is polymorphic in the human population with three different isoforms that are encoded by common alleles: E2, E3, and E4. ApoE3 is the “normal” isoform with an allele frequency of 78%. ApoE2 differs from ApoE3 based on a Cys residue at position 158 (allele frequency 7%) and is associated with Type III lipoproteinemia due to impaired binding to the LDL receptor (62). Type III hyperlipoproteinemia arising from rare as well as common ApoE variants could be corrected by APOE3 delivery, but levels would need to be maintained within a reasonable physiological range—i.e. not excessive overexpression. Therefore, in specific cases Type III hyperlipidemia is an excellent test case for targeted insertion into the Apoa1 locus. One can test whether APOE insertion can correct hyperlipidemia and atherosclerosis in the Apoe KO mice. The degree of atherosclerotic lesion formation is variable amongst mice, so these experiments may require n=15 per group. Apoe KO mice are maintained in house as a breeding colony. Mice are injected with AAV vectors at a dose of 5E11 GC per virus at 8 weeks of age. The groups may be as follows: 1) saline, 2) SaCas9/gRNA, 3) 2A-APOE, 4) 2A-APOE+SaCas9/gRNA. Animals are placed on a standard western type diet (21% fat, 0.15% cholesterol w/w, Research Diets D12079B). Plasma may be collected before injection and then at 2, 4, 6, 8, 12, and 16 weeks post-injection. The animals may be sacrificed at 16 weeks of age, for determination of atherosclerotic lesion burden. Atherosclerosis is assessed through en face staining of whole aortae, as well as H&E staining of ten micron paraffin sections of the aortic sinus. The Lagor laboratory has considerable published experience performing murine atherosclerosis studies (24,25,63). These measurements are performed in a blinded fashion, and independently verified by a second observer, using Image J software. The plasma levels of triglycerides, total cholesterol, HDL cholesterol, and non-HDL cholesterol may be measured enzymatically (24). The Apo E protein levels in the blood may be determined by ELISA over time.

One can achieve stable expression of human Apo E in plasma. Targeted insertion of APOE results in improved clearance of ApoB-containing lipoproteins, in specific embodiment. This may manifest as lower levels of triglycerides and cholesterol. In specific embodiments, as little as 5% restoration of APOE expression has a therapeutic effect. A statistically significant reduction in atherosclerotic lesion burden is evidence of disease correction ad may be achieved with methods of the disclosure.

In embodiments wherein the Apoa1 locus does not support high expression of transgenes, one can utilize a number of other highly expressed genes in the liver—HP, SAA1, SERPINA1, FGG, or APOA2, for example. In cases where the transgenes may be nonfunctional or secreted poorly with a 2A tag, one can instead use IRES, which preserves the native amino acid sequence. In cases wherein IRES elements are utilized, they may reduce the expression of the downstream transgene. In such cases, one could change the configuration of the Repair Cassette to insert the transgene of interest upstream of Apoa1. In cases where off-target editing with the gRNA is toxic or detrimental to the liver (though unlikely based on the bioinformatically predicted off-target sites that have a low degree of complementarity to the gRNA), one can utilize other choices for effective gRNA. With respect to persistent Cas9 expression needing to be avoided in the context of human gene therapy, AAV-CRISPR for these studies should address this. In some cases, nanoparticle delivery of Cas9 may be utilized, with the Repair Template still supplied by AAV. Additionally, a self-deleting AAV-CRISPR system may be utilized (27). In cases where there is significant off-target integration of the AAV-CRISPR vector or Repair cassette, one can address this with an unbiased analysis of vector genome integrations by LM-PCR. One can expect that the Apoa1 locus is a hotspot for NHEJ insertion with both vectors, but this should not be detrimental to the approach, as only one allele needs to be targeted correctly, and most hepatocytes are either 4n or 8n.

### Example 2

### Development a Flexible System for Selective Expansion of Gene-Targeted Cells

Targeted integration has the potential to achieve permanent expression of therapeutic transgenes in the liver. However, initial targeting rates are expected to be low, thus limiting this technology primarily to secreted proteins with a low threshold of correction (i.e. FIX, APOE). In order to make this technology universally applicable to liver diseases, this disclosure provides a system for selective expansion of the gene-targeted hepatocytes. The liver (as one example of a tissue for which the system may be utilized) has an incredible regenerative capacity, and can be completely replenished through proliferation of existing hepatocytes following a ⅔ partial hepatectomy (64). Thus, every hepatocyte in the liver has the capacity to divide, provided the correct stimulus is provided. In specific embodiments, one can metabolically injure hepatocytes through deletion of an essential gene with AAV-CRISPR or AAV-shRNA, for example. At the same time, cells with correct targeted integration into the Apoa1 locus carry a functional copy of the essential gene, along with the therapeutic transgene (FIG. 2). The targeted cells have a survival advantage and repopulate the liver at the expense of neighboring hepatocytes. The selection pressure in this system can be titrated both positively and negatively. Over time, the gene-targeted hepatocytes expand and repopulate the liver, ensuring each cell carries a permanent copy of the therapeutic transgene. In addition, the selectable marker is an endogenous gene, whose expression is ultimately restored in the expanded cells.

In specific embodiments of the disclosure, the following are examples of criteria for the vector system, and one or more may be applicable to the system:
1) The vector system promotes targeted integration into a common safe harbor site (i.e. Apoa1), which supports high expression of therapeutic transgenes.
2) Inducible hepatocyte injury is utilized to condition the liver for selective expansion. The injury in specific cases is generalizable and not specific to the disease to be corrected.
3) Exogenous genes are avoided as selectable markers (i.e. neomycin resistance), as permanent expression of these proteins is not desirable for human gene therapy.
4) In specific embodiments, the selection pressure is controllable, both positively and negatively, with either drugs or diet.
5) What is broken should also be replaced. The system should not generate a new genetic disease in order to rescue another. The gene-targeted hepatocytes support normal liver physiology, without increased susceptibility to other environmental insults (i.e. defects in drug export or catabolism).

The system of the disclosure involves using integration of an essential gene for selection of gene-targeted cells, such as hepatocytes. For initial studies, the fumarylacetoacetate hydrolase gene (Fah) is utilized, whose loss causes Hereditary Tyrosinemia Type I (OMIM: 276700). Loss of the Fah enzyme in the liver results in hepatocyte apoptosis and necrosis through accumulation of toxic tyrosine catabolites (65). To preserve hepatocyte viability in the absence of loss of FAH expression, mice can be maintained on a clinically approved drug, NTBC, which blocks the pathway upstream resulting in production of excretable catabolites (66,67). NTBC can be withdrawn as needed to apply selective pressure, which can be accelerated with a high protein diet. In specific cases, over a period of 3-6 months, correctly targeted hepatocytes expand leading to liver-wide restoration of the therapeutic transgene. In particular embodiments, this in vivo selection approach allows for treatment of any liver disease with targeted integration.

In specific embodiments, gene-corrected cells, such as hepatocytes, are selectively expanded through deletion of an essential gene, while simultaneously restoring its expression through precise integration.

In particular embodiments, the system of the disclosure is utilized with respect to FAH and Hereditary Tyrosinemia Type I (HT-I). Fumarylacetoacetate hydrolase (Fah) catalyzes the conversion of 4-fumarylacetoacetate to acetoacetate and fumarate. This enzyme is highly expressed in the liver where it is responsible for the final step in tyrosine catabolism. Loss-of-function mutations in the human FAH gene underlie an autosomal recessive genetic disease known as Hereditary Tyrosinemia Type I (HT-I) (OMIM 276700). Patients with HT-I present with severe liver failure in the neonatal period, requiring liver transplantation. Toxic metabolites accumulate in the absence of FAH activity (i.e. succinylacetone) which cause hepatocyte apoptosis, necrosis, and repeated cycles of liver regeneration. If untreated, liver injury will progress to cirrhosis and hepatocellular carcinoma and death at an early age. In 1992, Lindstedt et al. discovered that these patients could be treated with 2-(2-nitro-4-trifluoromethylbenzoyl)-1,3-cyclohexanedione (NTBC)(68). This drug is an inhibitor of an upstream enzyme, 4-Hydroxylphenyl-pyruvate Dioxygenase (HPD), which converts 3-(4-hydroxyphenyl)pyruvate to homogentisate. The products of this reaction are considerably less reactive and can be excreted in the urine (38). Thus, treatment with NTBC involves metabolic rerouting that preserves the health of the liver, essentially converting HT-I to a far more benign phenotype.

Selective expansion of Fah+ cells. Grompe et al. found that mice with homozygous deficiency of Fah are lethal in the neonatal period, but can be rescued by supplementation of NTBC to the drinking water (69). It has also been shown that transplantation of ˜1,000 Fah+ hepatocytes are sufficient to rescue the disease through repopulation of the liver (70). These findings provided the basis for the FRG humanized mouse model, in which human hepatocytes can be transplanted into immunodeficient mice lacking Fah (34,71-73). Over time, the human hepatocytes can repopulate up to 95% of the murine liver. In this model, the animals are maintained on NTBC to preserve liver health. NTBC can then be withdrawn, or “cycled,” in short 2-3 week increments to induce damage of the Fah-deficient murine hepatocytes. Over time, the transplanted Fah+ human hepatocytes have a survival advantage, and repopulate the liver. In recent years, genome editing has also been used to correct HT-I in the Fah KO mice using transposon insertion (74), Adenoviral gene therapy (75), AAV-mediated homologous recombination (76), CRISPR/Cas9 editing (77), and even base editors (78). In all cases, the corrected cells have a strong growth advantage and restore the liver over a period of several months. Thus, HT-I is an example of a genetic liver disease with a low threshold of correction, where even a small degree of editing (1-5%) can restore liver function. As such, it is useful to characterize the present approach, which couples an essential gene to transgene insertion.

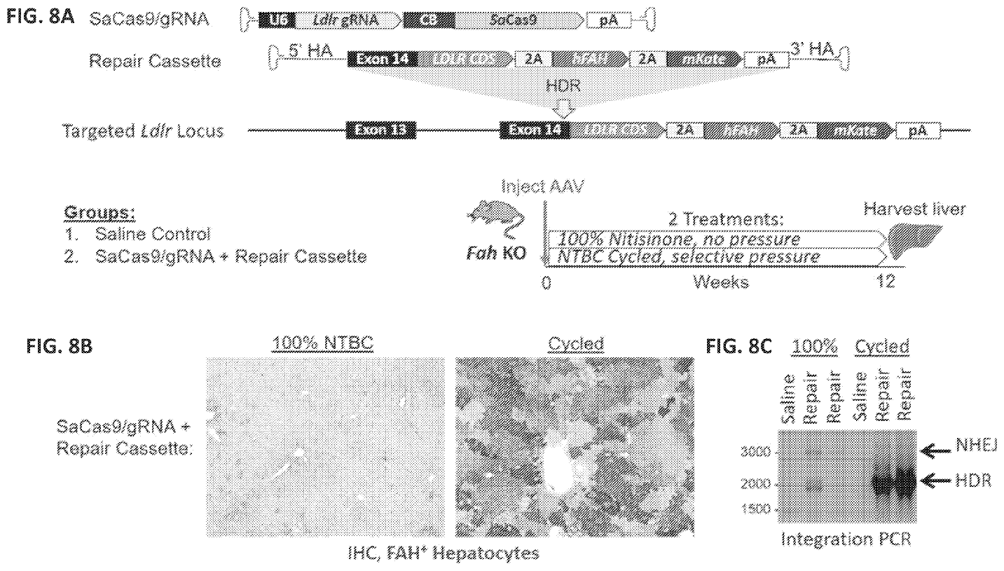

FAH as a selectable marker to expand genome-edited hepatocytes. To examine the feasibility of using FAH for positive selection in the liver, AAV vectors were generated expressing SaCas9 and a gRNA targeting the Ldlr gene. The low density lipoprotein receptor (Ldlr) is responsible for clearance of ApoB lipoproteins from the circulation, and loss-of-function mutations in this gene cause Familial Hypercholesterolemia (OMIM 143890). The gRNA targets Exon 14 of the Ldlr gene, and was designed to promote targeted integration of the remainder of the Ldlr coding sequence (CDS). In this case, the AAV repair template includes homology arms, the remainder of the Ldlr CDS, fused to a 2A skipping peptide, human FAH cDNA, followed by another 2A, an mKate2 reporter gene, and poly A signal (FIG. 8A). Correct integration of this repair cassette through HDR is expected to restore Ldlr expression, and also allow for expansion of these cells that also express FAH. To further characterize this, female adult Fah KO mice were injected with both AAV vectors at a dose of 5E11 GC each. Half of these animals were maintained on 100% NTBC in the drinking water for the entire study (uncycled), while the other half were cycled on and off NTBC (cycled) to apply selective pressure. Twelve weeks later, the mice were sacrificed and livers were harvested for analysis. Immunostaining for the FAH selectable marker was performed on paraffin sections from these livers. The mice in the uncycled group (100% NTBC, no selective pressure) showed rare individual hepatocytes with FAH expression (FIG. 8B). In contrast, the cycled group (NTBC cycling, strong selective pressure) had impressive outgrowth of colonies of FAH+ hepatocytes. PCR was used to detect the relative proportion of NHEJ insertions of the AAV genome versus correct HDR integrations. The uncycled mice had modest but detectable amounts of NHEJ and HDR events, as expected from the low frequency of FAH+ hepatocytes without selection. The cycled mice however had an overwhelming amount of HDR relative to NHEJ insertions, strongly supporting the competence of hepatocytes with FAH transgene integration to expand (FIG. 8C). This data supports the embodiment of using an essential gene as a selectable marker for expansion of gene-targeted hepatocytes.

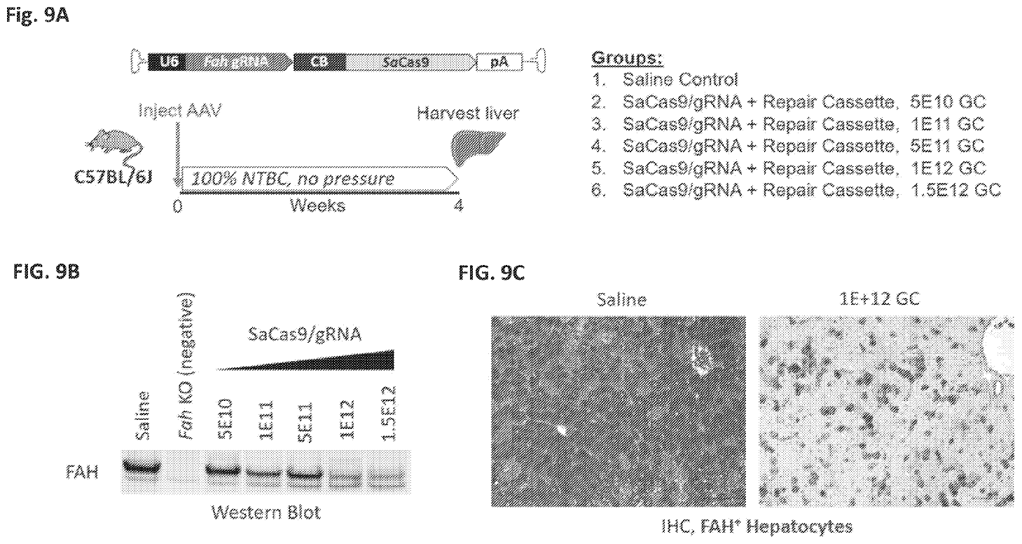

Optimizing FAH disruption as the “poison pill” for selection. The previous data was acquired in the Fah KO mice, where the entire liver is completely deficient in this enzyme. To make this approach generalizable to gene therapy patients, the essential gene (i.e. Fah) is removed efficiently in the rest of the liver to allow for selective expansion. It was next tested whether it is possible to remove Fah from the liver using an AAV-CRISPR vector. Wild type C57BL6/J mice were injected with AAV vectors encoding SaCas9 and a gRNA targeting Fah at doses of 5×10¹⁰, 1×10¹¹, 5×10¹¹, 1×10¹², and 1.5×¹² GC per mouse. The animals were maintained on 100% NTBC to prevent any injury or selection, and then sacrificed one month later (FIG. 9A). Efficient and dose-dependent removal of Fah was achieved based on western blotting for the Fah protein (FIG. 9B). Removal appeared maximal at 1×10¹² GC/mouse (FIG. 9C).

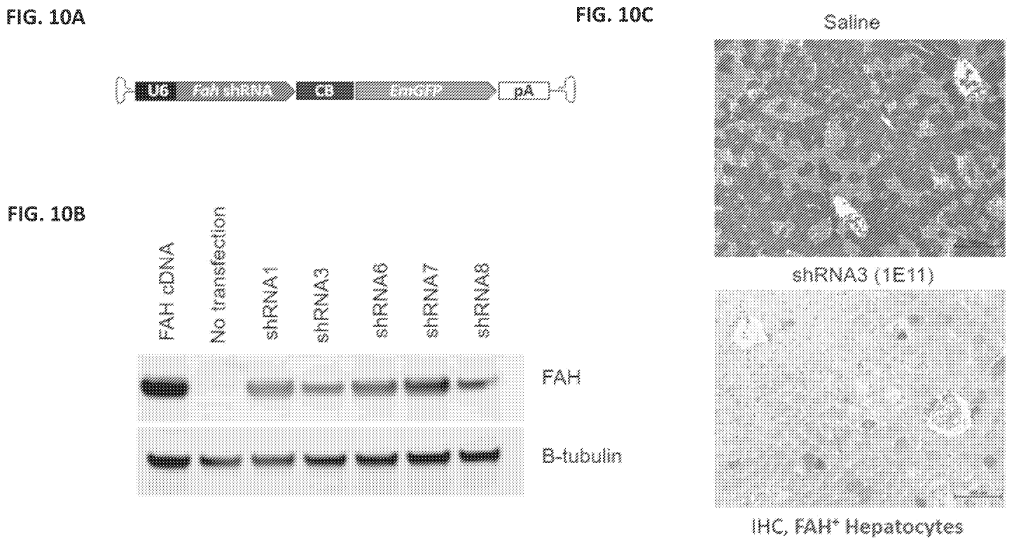

AAV-shRNA targeting FAH. As a complementary approach for Fah removal AAV vectors expressing an shRNA to this gene driven by the U6 promoter were constructed (FIG. 10A). These AAV plasmids were tested in HEK293T cells for knockdown efficiency by co-transfection with an expression vector for murine Fah. Several shRNA sequences were capable of Fah knockdown, with shRNA3 appearing to be the most potent (FIG. 10B). When packaged into AAV8, shRNA3 is also capable of efficient Fah knockdown in the liver, following 1 month on 100% NTBC (FIG. 10C).

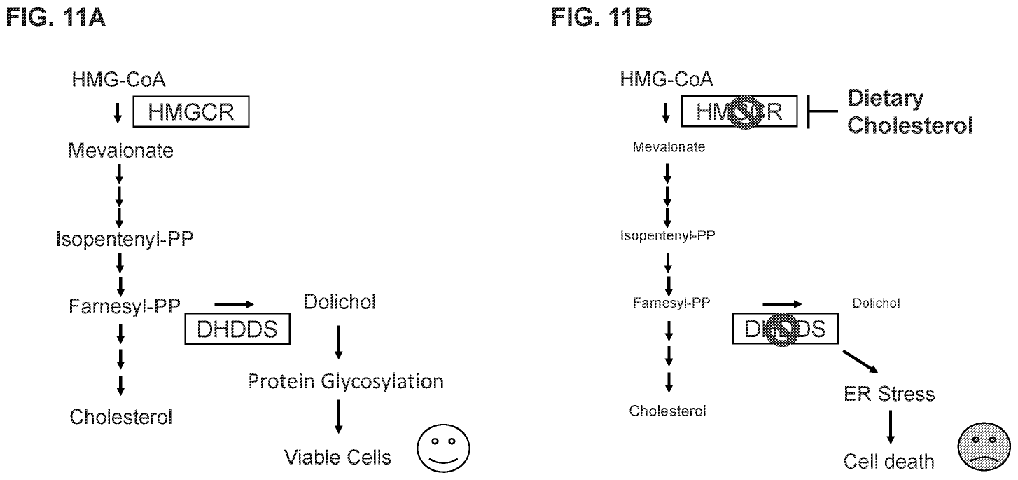

DHDDS, another essential gene for selection. In another example, dehydrodolichyl diphosphate synthase subunit (DHDDS), is the essential gene used to provide a selective advantage for the targeted hepatocytes. DHDDS is a component of the dehydrodolichol diphosphate synthase complex, which catalyzes the cis-prenyl chain elongation to produce dolichol diphosphate. Dehydrodolichol diphosphate is a sugar carrier involved in the synthesis of complex carbohydrates in the endoplasmic reticulum (ER) prior to their transfer to proteins. Loss of DHDDS activity will inhibit both N- and O-linked glycosylation, resulting in severe ER stress and cell death. The substrates for DHDDS are isopentenyl pyrophosphate and farnesyl pyrophosphate, derived from the mevalonate pathway. The mevalonate pathway also produces cholesterol, and is subject to stringent feedback inhibition by cholesterol (FIG. 11). This occurs at the level of 3-hydroxy-3-methylglutaryl Co enzyme A reductase (HMGCR), the rate limiting enzyme and target of the statin drugs. HMGCR is degraded in the presence of excess cholesterol. Cholesterol supplementation to the diet has been shown to potently suppress dolichol synthesis, and can be used to in conjunction with DHDDS inhibition to induce hepatocyte death.

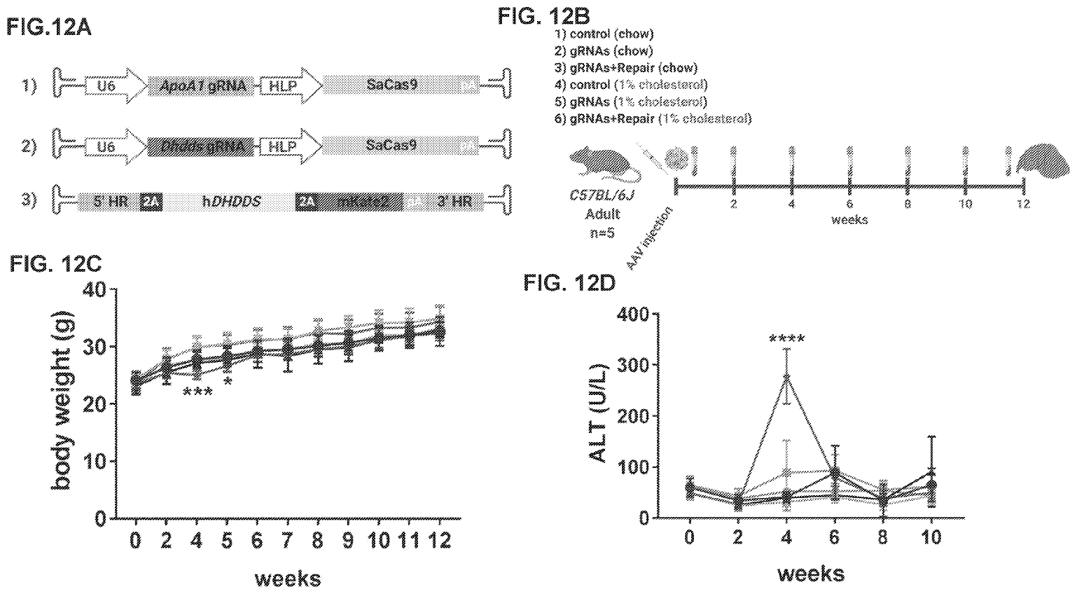

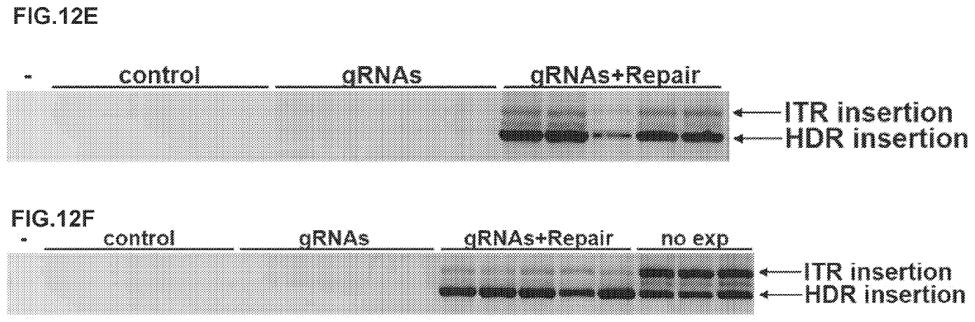

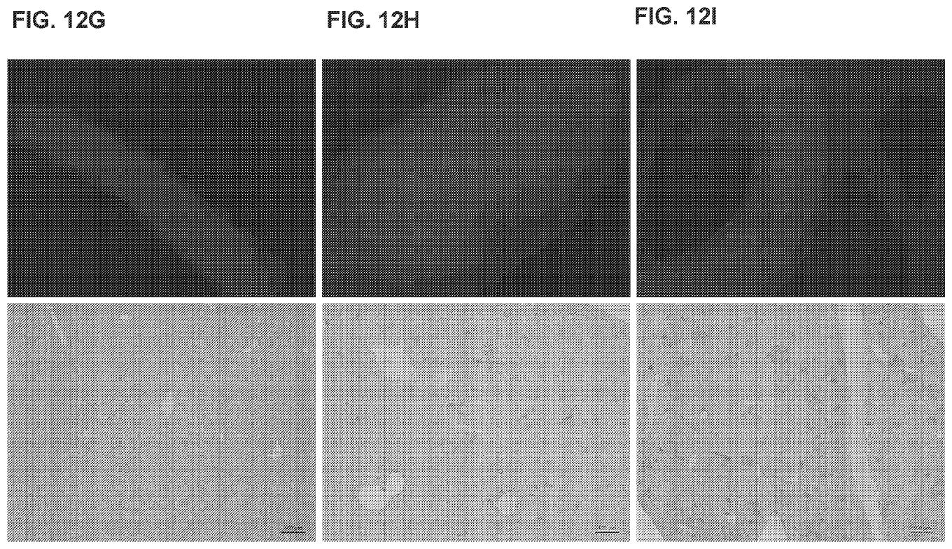

To test this concept, the inventors designed an experiment where a DHDDS transgene is used as a selectable marker with AAV-mediated genome editing. Mice were treated with AAV vectors encoding CRISPR/Cas9 and guide RNAs targeting both the Apoa1 gene (safe harbor locus), as well as the mouse Dhdds gene (essential gene). In addition, a third AAV vector supplies a repair template that can integrate at the Apoa1 locus through homologous recombination. This repair template contains the remainder of the murine Apoa1 coding sequence, a 2A peptide, a human DHDDS transgene, another 2A peptide, and an mKate2 fluorescent reporter (FIG. 12A). Mice were injected with either saline (control), both AAV-CRISPR vectors (gRNA only), or both AAV-CRISPR vectors and the repair template (gRNAs+repair). One group was maintained on a normal chow diet lacking cholesterol. The second group was placed on a diet containing 1% cholesterol (w/w) to apply further selective pressure to cells with deletion of Dhdds. Mice were followed for 12 weeks after AAV administration to determine the effects on body weight, transaminases, integration, and selective expansion of gene-corrected hepatocytes (FIG. 12B). Body weights were comparable between the groups, with the exception of a transient drop at 4 and 5 weeks for mice that received both AAV-CRISPR vectors and the 1% cholesterol diet, consistent with Dhdds-dependent liver injury (FIG. 12C). This was also accompanied by a spike in alanine aminotransferase (ALT) activity in the plasma, indicative of liver damage. Mice receiving both AAV-CRISPR vectors and the repair template did not have significant changes in body weight or liver enzyme elevations, indicating protection provided by the integrated transgene cassette (FIG. 12D). Integration PCR of the Apoa1 locus revealed two major products—a) a higher band corresponding to ligation of the entire AAV repair cassette at the CRISPR cut site, termed ITR insertion, and b) the correct homology directed repair product (HDR). Integration was only detectable in the groups receiving both AAV-CRISPR vectors and the repair cassette (FIGS. 12E and 12F). The relative intensity of the HDR band was greater in the group fed 1% cholesterol. The ratio of the HDR:ITR band, indicative of correct repair and expansion, also exceeded that of positive control samples in the last three lanes from mice without the selectable marker or Dhdds deletion (FIG. 12F). Targeting frequency and transgene expression was confirmed by direct fluorescence to detect the mKate2 reporter (FIGS. 12G-12I), as well as immunohistochemstry for a flag epitope tag on mKate2. Colonies of positive cells are clearly visible in the image at the right from mice fed the 1% cholesterol diet, indicating selective expansion of gene-corrected cells with the dietary manipulation.

Experimental Design

Test if the ApoA1 locus can support selective expansion in Fah KO mice. In initial data, there is evidence that knocking in the Fah CDS can be used to selectively expand gene-targeted hepatocytes. In this study, one can determine if targeted integration into the Apoa1 locus can support selective expansion. To accomplish this, one can modify the AAV repair template for Apoa1. This vector can include the final coding exon of Apoa1, fused to a 2A skipping peptide, FAH, another 2A sequence, followed by an mKate2 reporter gene. Mice with germline deficiency of Fah may be used (Fah KO) to eliminate confounding variables related to Fah knockdown efficiency. The groups (n=8) may be as follows: 1) saline, 2) Apoa1-FAH repair only, 3) Apoa1-FAH repair+SaCas9/Apoa1 gRNA. All the mice may be kept on 100% NTBC until AAV injection, and then split into two groups thereafter: a) uncycled and b) cycled. Mice may be sacrificed 3 months later to allow time for selective expansion. In specific embodiments, the uncycled mice kept on 100% NTBC have expression of FAH and mKate2 that is reflective of the initial targeting rates—i.e. very low with repair cassette alone, and higher with repair cassette+CRISPR. In the mice that are cycled, in specific cases selective expansion of FAH+/mKate2+ hepatocytes in groups 2 and 3. In particular cases there are far bigger colonies in the livers of the mice in group 3, where AAV-CRISPR was used to open the Apoa1 locus for integration. A positive result from this study confirms proper configuration and expression competence of the repair template, as well as the ability of the Apoa1 locus to support selective expansion.

Compare the effectiveness of AAV-CRISPR to AAV-shRNA. Initial data shows that AAV-CRISPR and AAV-shRNA can both significantly reduce Fah levels in the liver. In this study, one can compare the two approaches for Fah removal in terms of their ability to promote selective expansion. One can use the most effective gRNA and shRNA identified above. Groups of C57BL6/J mice (n=16) are injected with either: 1) Saline (negative control), 2) Apoa1-2A-FAH-2A-mKate2 repair template, 3) repair template+AAV-shRNA, or 4) repair template+AAV-CRISPR. In addition, half of the mice in each group (n=8) are maintained on 100% NTBC where there is no selective pressure. The other half of the mice (n=8) are cycled on and off NTBC to promote expansion. In specific cases, for clarity, all mice without NTBC and with shRNA or CRISPR against Fah undergo apoptosis due to accumulation of succinylacetone, while integrated repair cassette containing FAH should be able to rescue this lethal phenotype and lead to clonal expansion (selection advantage). Three months later, mice are sacrificed for liver harvest. The primary readouts are mKate2 expression by western blotting and immunostaining for the FLAG epitope tag on this protein. In addition, PCR is used to assess the relative frequency of NHEJ insertions versus HDR events. In specific embodiments, both the AAV-shRNA and the AAV-CRISPR approaches succeed in promoting selective expansion of Apoa1-targeted hepatocytes. In specific embodiments, there are more mKate2+ cells in each of these groups (3 and 4), relative to animals receiving the repair template alone (group 2). The most effective approach may be carried forward to assess the durability of expression below.

Test the effectiveness and durability of therapeutic transgene expression with selective expansion. In this study, one can examine the durability of therapeutic transgene expression. In specific cases, AAV-CRISPR is used to delete Fah, although one can proceed with AAV-shRNA if more effective expansion of mKate2+ cells is obtained (see above). For this study, AAV repair templates are built to include the secreted proteins APOE or FIX. These are combined with the human FAH selectable marker (i.e. Apoa1-2A-APOE-2A-FAH-pA or Apoa1-2A-FIX-2A-FAH-pA). These transgenes are therapeutically relevant and also allow for longitudinal monitoring of protein levels in the blood, which should reflect the expansion of corrected cells. Groups of C57BL6/J mice (n=30) are injected with either: 1) Saline (negative control), 2) SaCas9/gRNA (to both Apoa1 and Fah), 3) Repair cassette alone, 4) Repair cassette+SaCas9/gRNA (to both Apoa1 and Fah). Following injection the groups are split, with half of the mice in each group (n=15) maintained on 100% NTBC. The other half of the mice in each group (n=15) is cycled on and off NTBC. The large numbers per group (n=15) are necessary to establish the safety of the approach, described below. Plasma is collected before AAV injection, and then at 1, 2, 3, 6, 9, and 12 months thereafter. The mice are sacrificed at 12 months after AAV administration to harvest livers for analysis. The levels of FIX and APOE in the plasma are determined by ELISA. One can also monitor the production of ApoA1-2A in the plasma by western blotting for the 2A tag as a readout of site-specific integration. In particular embodiments, there is detectable expression of FIX and APOE in the plasma of mice injected with the Repair Cassette alone and maintained on 100% NTBC. Higher levels of FIX and APOE are seen in the mice treated with AAV-CRISPR because of more efficient integration, in specific embodiments. In both cases, the groups cycled on and off NTBC have significant increases in FIX and APOE in the plasma that increase steadily over time, in particular embodiments.

Assess the long-term safety of Repair Drive using FAH selection. The study described above involves longitudinal follow up over a 12-month period, in specific cases. In addition to monitoring transgene expression in the plasma, the degree of liver injury is determined by measuring transaminases (ALT and AST). The competence of the liver to secrete important plasma proteins may be assessed using ELISAs to fibrinogen as well as ApoA1 and ApoB. Animal health may be monitored continuously throughout the study, and a body weight drop of 15% results in conversion back to 100% NTBC until resolved. At the end of the 12 month study, entire livers are examined for tumors or preneoplastic nodules by taking 2-3 mm cross sections through the entirety of the organ with a razor blade. Any portion of a lobe with regions that deviate from normal appearance are fixed in formalin and sectioned. H&E staining is performed to identify tumors as well as pre-neoplastic growths. If these occur, the number of mice with tumors in each group are compared to the control group by Fisher's exact test. Possible fibrosis is assessed in paraffin sections by Sirius red staining. In addition, DNA may be isolated from livers for determination of on- and off-target editing with both gRNA's using targeted deep sequencing. The top 20 predicted off-target sites for each gRNA may be examined. In addition, an unbiased analysis of vector genome insertions may be performed by ligation-mediated PCR using a primer that recognizes either the ITR or internal sequences of the AAV vector. If tumors are observed, these would be carefully dissected for DNA isolation, and subjected to LM-PCR to define the relevant AAV integration sites underlying any tumorigenic event.

In particular embodiments, liver function as a whole is preserved throughout the course of the study, even in the setting of selection. This would be evident by normal levels of fibrinogen, ApoA1 and/or ApoB, for example. In specific cases, liver transaminases (ALT, AST) spike upon NTBC withdrawal, and this gradually resolves over time. Although there may be a low incidence of tumors in aged C57BL/6J mice, this may differ between the groups. If it does, one can identify the root cause through sequencing of off-target sites and AAV integration events.

In specific embodiments where the Apoa1 locus cannot support high enough expression of Fah for repopulation (which should be unlikely as Apoa1 is one of the highest expressed genes in the liver, far exceeding that of Ldlr, that was targeted and expanded successfully in initial data), one can switch to albumin targeting if needed. In some cases, murine cells escaping complete Fah deletion may compete with gene targeted cells for expansion. If this occurs, one can find a more efficient gRNA or shRNA. If this is still insufficient, AAV-CRISPR and AAV-shRNA may be used in combination to maximize Fah removal. In situations where high doses of AAV-shRNA are toxic to the liver as reported by Grimm et al. (79), one could use a lower dose, although it is also possible that this method could improve selection, as the AAV-shRNA genome would not integrate. Alternatively, less active Pol II-driven expression of shRNA could be used. In cases where cells may escape metabolic poisoning by Fah deletion because of inefficiencies in the single AAV delivery, one can utilize alternative strategies for Fah knockdown that can be dosed repeatedly, such as locked nucleic acids and GalNac-modified siRNA. 5) It is possible that in the possibility that Fah deletion will result in acute liver failure, this should not happen because animals are maintained on 100% NTBC until editing is complete, and then gradually cycled off the drug, with careful monitoring. If the mice may get tumors because of unintended off-target cutting or insertion of the AAV vector, one can pay careful attention to the tumors themselves, as any driver mutations would be clonally expanded. New hotspots for AAV integration would be identified by LM-PCR. One can also set up studies to determine whether or not insertion into Apoa1 itself carries any risk of tumorigenesis.

### REFERENCES

All publications mentioned in this specification are indicative of the level of those skilled in the art to which the invention pertains. All publications herein are incorporated by reference to the same extent as if each individual publication was specifically and individually indicated to be incorporated by reference in their entirety.
1. Cunniff, C., Carmack, J. L., Kirby, R. S., and Fiser, D. H. (1995) Contribution of heritable disorders to mortality in the pediatric intensive care unit. Pediatrics 95, 678-681
2. Rangarajan, S., Walsh, L., Lester, W., Perry, D., Madan, B., Laffan, M., Yu, H., Vettermann, C., Pierce, G. F., Wong, W. Y., and Pasi, K. J. (2017) AAV5-Factor VIII Gene Transfer in Severe Hemophilia A. N Engl J Med 377, 2519-2530
3. George, L. A., Sullivan, S. K., Giermasz, A., Rasko, J. E. J., Samelson-Jones, B. J., Ducore, J., Cuker, A., Sullivan, L. M., Majumdar, S., Teitel, J., McGuinn, C. E., Ragni, M. V., Luk, A. Y., Hui, D., Wright, J. F., Chen, Y., Liu, Y., Wachtel, K., Winters, A., Tiefenbacher, S., Arruda, V. R., van der Loo, J. C. M., Zelenaia, O., Takefman, D., Carr, M. E., Couto, L. B., Anguela, X. M., and High, K. A. (2017) Hemophilia B Gene Therapy with a High-Specific-Activity Factor IX Variant. N Engl J Med 377, 2215-2227
4. Nathwani, A. C., Reiss, U. M., Tuddenham, E. G., Rosales, C., Chowdary, P., McIntosh, J., Della Peruta, M., Lheriteau, E., Patel, N., Raj, D., Riddell, A., Pie, J., Rangarajan, S., Bevan, D., Recht, M., Shen, Y. M., Halka, K. G., Basner-Tschakarjan, E., Mingozzi, F., High, K. A., Allay, J., Kay, M. A., Ng, C. Y., Zhou, J., Cancio, M., Morton, C. L., Gray, J. T., Srivastava, D., Nienhuis, A. W., and Davidoff, A. M. (2014) Long-term safety and efficacy of factor IX gene therapy in hemophilia B. N Engl J Med 371, 1994-2004
5. Nathwani, A. C., Tuddenham, E. G., Rangarajan, S., Rosales, C., McIntosh, J., Linch, D. C., Chowdary, P., Riddell, A., Pie, A. J., Harrington, C., O'Beirne, J., Smith, K., Pasi, J., Glader, B., Rustagi, P., Ng, C. Y., Kay, M. A., Zhou, J., Spence, Y., Morton, C. L., Allay, J., Coleman, J., Sleep, S., Cunningham, J. M., Srivastava, D., Basner-Tschakarjan, E., Mingozzi, F., High, K. A., Gray, J. T., Reiss, U. M., Nienhuis, A. W., and Davidoff, A. M. (2011) Adenovirus-associated virus vector-mediated gene transfer in hemophilia B. N Engl J Med 365, 2357-2365
6. Mingozzi, F., and High, K. A. (2013) Immune responses to AAV vectors: overcoming barriers to successful gene therapy. Blood 122, 23-36
7. Schnepp, B. C., Jensen, R. L., Chen, C. L., Johnson, P. R., and Clark, K. R. (2005) Characterization of adeno-associated virus genomes isolated from human tissues. J Virol 79, 14793-14803
8. Magami, Y., Azuma, T., Inokuchi, H., Kokuno, S., Moriyasu, F., Kawai, K., and Hattori, T. (2002) Cell proliferation and renewal of normal hepatocytes and bile duct cells in adult mouse liver. Liver 22, 419-425
9. Macdonald, R. A. (1961) “Lifespan” of liver cells. Autoradio-graphic study using tritiated thymidine in normal, cirrhotic, and partially hepatectomized rats. Arch Intern Med 107, 335-343
10. Wang, L., Wang, H., Morizono, H., Bell, P., Jones, D., Lin, J., McMenamin, D., Yu, H., Batshaw, M. L., and Wilson, J. M. (2012) Sustained correction of OTC deficiency in spf(ash) mice using optimized self-complementary AAV2/8 vectors. Gene Ther 19, 404-410
11. Cancer Genome Atlas Research Network. Electronic address, w. b. e., and Cancer Genome Atlas Research, N. (2017) Comprehensive and Integrative Genomic Characterization of Hepatocellular Carcinoma. Cell 169, 1327-1341 e1323
12. Fujimoto, A., Furuta, M., Totoki, Y., Tsunoda, T., Kato, M., Shiraishi, Y., Tanaka, H., Taniguchi, H., Kawakami, Y., Ueno, M., Gotoh, K., Ariizumi, S., Wardell, C. P., Hayami, S., Nakamura, T., Aikata, H., Arihiro, K., Boroevich, K. A., Abe, T., Nakano, K., Maejima, K., Sasaki-Oku, A., Ohsawa, A., Shibuya, T., Nakamura, H., Hama, N., Hosoda, F., Arai, Y., Ohashi, S., Urushidate, T., Nagae, G., Yamamoto, S., Ueda, H., Tatsuno, K., Ojima, H., Hiraoka, N., Okusaka, T., Kubo, M., Marubashi, S., Yamada, T., Hirano, S., Yamamoto, M., Ohdan, H., Shimada, K., Ishikawa, O., Yamaue, H., Chayama, K., Miyano, S., Aburatani, H., Shibata, T., and Nakagawa, H. (2016) Whole-genome mutational landscape and characterization of noncoding and structural mutations in liver cancer. Nat Genet 48, 500-509
13. Schulze, K., Imbeaud, S., Letouze, E., Alexandrov, L. B., Calderaro, J., Rebouissou, S., Couchy, G., Meiller, C., Shinde, J., Soysouvanh, F., Calatayud, A. L., Pinyol, R., Pelletier, L., Balabaud, C., Laurent, A., Blanc, J. F., Mazzaferro, V., Calvo, F., Villanueva, A., Nault, J. C., Bioulac-Sage, P., Stratton, M. R., Llovet, J. M., and Zucman-Rossi, J. (2015) Exome sequencing of hepatocellular carcinomas identifies new mutational signatures and potential therapeutic targets. Nat Genet 47, 505-511
14. Villiger, L., Grisch-Chan, H. M., Lindsay, H., Ringnalda, F., Pogliano, C. B., Allegri, G., Fingerhut, R., Haberle, J., Matos, J., Robinson, M. D., Thony, B., and Schwank, G. (2018) Treatment of a metabolic liver disease by in vivo genome base editing in adult mice. Nat Med 24, 1519-1525
15. Shen, S., Sanchez, M. E., Blomenkamp, K., Corcoran, E. M., Marco, E., Yudkoff, C. J., Jiang, H., Teckman, J. H., Bumcrot, D., and Albright, C. F. (2018) Amelioration of Alpha-1 Antitrypsin Deficiency Diseases with Genome Editing in Transgenic Mice. Hum Gene Ther 29, 861-873
16. Song, C. Q., Wang, D., Jiang, T., O'Connor, K., Tang, Q., Cai, L., Li, X., Weng, Z., Yin, H., Gao, G., Mueller, C., Flotte, T. R., and Xue, W. (2018) In Vivo Genome Editing Partially Restores Alpha1-Antitrypsin in a Murine Model of AAT Deficiency. Hum Gene Ther 29, 853-860
17. Nygaard, S., Barzel, A., Haft, A., Major, A., Finegold, M., Kay, M. A., and Grompe, M. (2016) A universal system to select gene-modified hepatocytes in vivo. Sci Transl Med 8, 342ra379
18. Bortolussi, G., Zentillin, L., Vanikova, J., Bockor, L., Bellarosa, C., Mancarella, A., Vianello, E., Tiribelli, C., Giacca, M., Vitek, L., and Muro, A. F. (2014) Life-long correction of hyperbilirubinemia with a neonatal liver-specific AAV-mediated gene transfer in a lethal mouse model of Crigler-Najjar Syndrome. Hum Gene Ther 25, 844-855
19. Bissig-Choisat, B., Wang, L., Legras, X., Saha, P. K., Chen, L., Bell, P., Pankowicz, F. P., Hill, M. C., Barzi, M., Leyton, C. K., Leung, H. C., Kruse, R. L., Himes, R. W., Goss, J. A., Wilson, J. M., Chan, L., Lagor, W. R., and Bissig, K. D. (2015) Development and rescue of human familial hypercholesterolaemia in a xenograft mouse model. Nat Commun 6, 7339
20. Kassim, S. H., Li, H., Bell, P., Somanathan, S., Lagor, W., Jacobs, F., Billheimer, J., Wilson, J. M., and Rader, D. J. (2013) Adeno-associated virus serotype 8 gene therapy leads to significant lowering of plasma cholesterol levels in humanized mouse models of homozygous and heterozygous familial hypercholesterolemia. Hum Gene Ther 24, 19-26
21. Lagor, W. R., Brown, R. J., Toh, S. A., Millar, J. S., Fuki, I. V., de la Llera-Moya, M., Yuen, T., Rothblat, G., Billheimer, J. T., and Rader, D. J. (2009) Overexpression of apolipoprotein F reduces HDL cholesterol levels in vivo. Arterioscler Thromb Vasc Biol 29, 40-46
22. Lagor, W. R., Johnston, J. C., Lock, M., Vandenberghe, L. H., and Rader, D. J. (2013) Adeno-associated viruses as liver-directed gene delivery vehicles: focus on lipoprotein metabolism. Methods Mol Biol 1027, 273-307
23. O'Neill, S. M., Hinkle, C., Chen, S. J., Sandhu, A., Hovhannisyan, R., Stephan, S., Lagor, W. R., Ahima, R. S., Johnston, J. C., and Reilly, M. P. (2014) Targeting adipose tissue via systemic gene therapy. Gene Ther 21, 653-661
24. Jarrett, K. E., Lee, C., De Giorgi, M., Hurley, A., Gillard, B. K., Doerfler, A. M., Li, A., Pownall, H. J., Bao, G., and Lagor, W. R. (2018) Somatic Editing of Ldlr With Adeno-Associated Viral-CRISPR Is an Efficient Tool for Atherosclerosis Research. Arterioscler Thromb Vasc Biol 38, 1997-2006
25. Jarrett, K. E., Lee, C. M., Yeh, Y. H., Hsu, R. H., Gupta, R., Zhang, M., Rodriguez, P. J., Lee, C. S., Gillard, B. K., Bissig, K. D., Pownall, H. J., Martin, J. F., Bao, G., and Lagor, W. R. (2017) Somatic genome editing with CRISPR/Cas9 generates and corrects a metabolic disease. Sci Rep 7, 44624
26. Pan, X., Philippen, L., Lahiri, S. K., Lee, C., Park, S. H., Word, T. A., Li, N., Jarrett, K. E., Gupta, R., Reynolds, J. O., Lin, J., Bao, G., Lagor, W. R., and Wehrens, X. H. T. (2018) In Vivo Ryr2 Editing Corrects Catecholaminergic Polymorphic Ventricular Tachycardia. Circ Res 123, 953-963
27. Li, A., Lee, C. M., Hurley, A. E., Jarrett, K. E., De Giorgi, M., Lu, W., Balderrama, K. S., Doerfler, A. M., Deshmukh, H., Ray, A., Bao, G., and Lagor, W. R. (2019) A Self-Deleting AAV-CRISPR System for In Vivo Genome Editing. Mol Ther Methods Clin Dev 12, 111-122
28. Lin, Y., Cradick, T. J., Brown, M. T., Deshmukh, H., Ranjan, P., Sarode, N., Wile, B. M., Vertino, P. M., Stewart, F. J., and Bao, G. (2014) CRISPR/Cas9 systems have off-target activity with insertions or deletions between target DNA and guide RNA sequences. Nucleic Acids Res 42, 7473-7485
29. Cradick, T. J., Fine, E. J., Antico, C. J., and Bao, G. (2013) CRISPR/Cas9 systems targeting beta-globin and CCR5 genes have substantial off-target activity. Nucleic Acids Res 41, 9584-9592
30. Aouida, M., Eid, A., Ali, Z., Cradick, T., Lee, C., Deshmukh, H., Atef, A., AbuSamra, D., Gadhoum, S. Z., Merzaban, J., Bao, G., and Mahfouz, M. (2015) Efficient fdCas9 Synthetic Endonuclease with Improved Specificity for Precise Genome Engineering. PloS one 10, e0133373
31. Lee, C. M., Cradick, T. J., and Bao, G. (2016) The Neisseria meningitidis CRISPR-Cas9 System Enables Specific Genome Editing in Mammalian Cells. Mol Ther 24, 645-654
32. Zhu, H., Zhang, L., Tong, S., Lee, C. M., Deshmukh, H., and Bao, G. (2019) Spatial control of in vivo CRISPR-Cas9 genome editing via nanomagnets. Nat Biomed Eng 3, 126-136
33. Cradick, T. J., Qiu, P., Lee, C. M., Fine, E. J., and Bao, G. (2014) COSMID: A Web-based Tool for Identifying and Validating CRISPR/Cas Off-target Sites. Mol Ther Nucleic Acids 3, e214
34. Bissig, K. D., Le, T. T., Woods, N. B., and Verma, I. M. (2007) Repopulation of adult and neonatal mice with human hepatocytes: a chimeric animal model. Proc Natl Acad Sci USA 104, 20507-20511
35. Bissig-Choisat, B., Wang, L., Legras, X., Saha, P. K., Chen, L., Bell, P., Pankowicz, F. P., Hill, M. C., Barzi, M., Kettlun Leyton, C., Leung, H. C., Kruse, R. L., Himes, R. W., Goss, J. A., Wilson, J. M., Chan, L., Lagor, W. R., and Bissig, K. D. (2015) Development and rescue of human familial hypercholesterolaemia in a xenograft mouse model. Nat Commun 6, 7339
36. Barzi, M., Pankowicz, F. P., Zorman, B., Liu, X., Legras, X., Yang, D., Borowiak, M., Bissig-Choisat, B., Sumazin, P., Li, F., and Bissig, K. D. (2017) A novel humanized mouse lacking murine P450 oxidoreductase for studying human drug metabolism. Nat Commun 8, 39
37. Pankowicz, F. P., Barzi, M., Kim, K. H., Legras, X., Martins, C. S., Wooton-Kee, C. R., Lagor, W. R., Marini, J. C., Elsea, S. H., Bissig-Choisat, B., Moore, D. D., and Bissig, K. D. (2018) Rapid Disruption of Genes Specifically in Livers of Mice Using Multiplex CRISPR/Cas9 Editing. Gastroenterology 155, 1967-1970 e1966
38. Pankowicz, F. P., Barzi, M., Legras, X., Hubert, L., Mi, T., Tomolonis, J. A., Ravishankar, M., Sun, Q., Yang, D., Borowiak, M., Sumazin, P., Elsea, S. H., Bissig-Choisat, B., and Bissig, K. D. (2016) Reprogramming metabolic pathways in vivo with CRISPR/Cas9 genome editing to treat hereditary tyrosinaemia. Nat Commun 7, 12642
39. Pankowicz, F. P., Jarrett, K. E., Lagor, W. R., and Bissig, K. D. (2017) CRISPR/Cas9: at the cutting edge of hepatology. Gut 66, 1329-1340
40. Bissig-Choisat, B., Wang, L., Legras, X., Saha, P. K., Chen, L., Bell, P., Pankowicz, F. P., Hill, M. C., Barzi, M., Leyton, C. K., Leung, H. E., Kruse, R. L., Himes, R. W., Goss, J. A., Wilson, J. M., Chan, L., Lagor, W. R., and Bissig, K. D. (2015) Development and rescue of human familial hypercholesterolaemia in a xenograft mouse model. Nat Commun 6, 7339
41. Samulski, R. J., Zhu, X., Xiao, X., Brook, J. D., Housman, D. E., Epstein, N., and Hunter, L. A. (1991) Targeted integration of adeno-associated virus (AAV) into human chromosome 19. EMBO J 10, 3941-3950
42. Nault, J. C., Mami, I., La Bella, T., Datta, S., Imbeaud, S., Franconi, A., Mallet, M., Couchy, G., Letouze, E., Pilati, C., Verret, B., Blanc, J. F., Balabaud, C., Calderaro, J., Laurent, A., Letexier, M., Bioulac-Sage, P., Calvo, F., and Zucman-Rossi, J. (2016) Wild-type AAV Insertions in Hepatocellular Carcinoma Do Not Inform Debate Over Genotoxicity Risk of Vectorized AAV. Mol Ther 24, 660-661.
43. Barzel, A., Paulk, N. K., Shi, Y., Huang, Y., Chu, K., Zhang, F., Valdmanis, P. N., Spector, L. P., Porteus, M. H., Gaensler, K. M., and Kay, M. A. (2015) Promoterless gene targeting without nucleases ameliorates haemophilia B in mice. Nature 517, 360-364
44. Borel, F., Tang, Q., Gernoux, G., Greer, C., Wang, Z., Barzel, A., Kay, M. A., Shultz, L. D., Greiner, D. L., Flotte, T. R., Brehm, M. A., and Mueller, C. (2017) Survival Advantage of Both Human Hepatocyte Xenografts and Genome-Edited Hepatocytes for Treatment of alpha-1 Antitrypsin Deficiency. Mol Ther 25, 2477-2489
45. Porro, F., Bortolussi, G., Barzel, A., De Caneva, A., Iaconcig, A., Vodret, S., Zentilin, L., Kay, M. A., and Muro, A. F. (2017) Promoterless gene targeting without nucleases rescues lethality of a Crigler-Najjar syndrome mouse model. EMBO Mol Med 9, 1346-1355
46. Sharma, R., Anguela, X. M., Doyon, Y., Wechsler, T., DeKelver, R. C., Sproul, S., Paschon, D. E., Miller, J. C., Davidson, R. J., Shivak, D., Zhou, S., Rieders, J., Gregory, P. D., Holmes, M. C., Rebar, E. J., and High, K. A. (2015) In vivo genome editing of the albumin locus as a platform for protein replacement therapy. Blood 126, 1777-1784
47. Laoharawee, K., DeKelver, R. C., Podetz-Pedersen, K. M., Rohde, M., Sproul, S., Nguyen, H. O., Nguyen, T., St Martin, S. J., Ou, L., Tom, S., Radeke, R., Meyer, K. E., Holmes, M. C., Whitley, C. B., Wechsler, T., and McIvor, R. S. (2018) Dose-Dependent Prevention of Metabolic and Neurologic Disease in Murine MPS II by ZFN-Mediated In Vivo Genome Editing. Mol Ther 26, 1127-1136
48. Ou, L., DeKelver, R. C., Rohde, M., Tom, S., Radeke, R., St Martin, S. J., Santiago, Y., Sproul, S., Przybilla, M. J., Koniar, B. L., Podetz-Pedersen, K. M., Laoharawee, K., Cooksley, R. D., Meyer, K. E., Holmes, M. C., McIvor, R. S., Wechsler, T., and Whitley, C. B. (2019) ZFN-Mediated In Vivo Genome Editing Corrects Murine Hurler Syndrome. Mol Ther 27, 178-187
49. Cradick, T. J., Antico, C. J., and Bao, G. (2014) High-throughput cellular screening of engineered nuclease activity using the single-strand annealing assay and luciferase reporter. Methods Mol Biol 1114, 339-352
50. McIntosh, J., Lenting, P. J., Rosales, C., Lee, D., Rabbanian, S., Raj, D., Patel, N., Tuddenham, E. G., Christophe, O. D., McVey, J. H., Waddington, S., Nienhuis, A. W., Gray, J. T., Fagone, P., Mingozzi, F., Zhou, S. Z., High, K. A., Cancio, M., Ng, C. Y., Zhou, J., Morton, C. L., Davidoff, A. M., and Nathwani, A. C. (2013) Therapeutic levels of FVIII following a single peripheral vein administration of rAAV vector encoding a novel human factor VIII variant. Blood 121, 3335-3344
51. Xiao, X., Li, J., and Samulski, R. J. (1998) Production of high-titer recombinant adeno-associated virus vectors in the absence of helper adenovirus. J Virol 72, 2224-2232
52. Gil-Farina, I., Fronza, R., Kaeppel, C., Lopez-Franco, E., Ferreira, V., D'Avola, D., Benito, A., Prieto, J., Petry, H., Gonzalez-Aseguinolaza, G., and Schmidt, M. (2016) Recombinant AAV Integration Is Not Associated With Hepatic Genotoxicity in Nonhuman Primates and Patients. Mol Ther 24, 1100-1105
53. Chandler, R. J., LaFave, M. C., Varshney, G. K., Trivedi, N. S., Carrillo-Carrasco, N., Senac, J. S., Wu, W., Hoffmann, V., Elkahloun, A. G., Burgess, S. M., and Venditti, C. P. (2015) Vector design influences hepatic genotoxicity after adeno-associated virus gene therapy. J Clin Invest 125, 870-880
54. Nault, J. C., Datta, S., Imbeaud, S., Franconi, A., Mallet, M., Couchy, G., Letouze, E., Pilati, C., Verret, B., Blanc, J. F., Balabaud, C., Calderaro, J., Laurent, A., Letexier, M., Bioulac-Sage, P., Calvo, F., and Zucman-Rossi, J. (2015) Recurrent AAV2-related insertional mutagenesis in human hepatocellular carcinomas. Nat Genet 47, 1187-1193
55. Kuwano, T., Bi, X., Cipollari, E., Yasuda, T., Lagor, W. R., Szapary, H. J., Tohyama, J., Millar, J. S., Billheimer, J. T., Lyssenko, N. N., and Rader, D. J. (2017) Overexpression and deletion of phospholipid transfer protein reduce HDL mass and cholesterol efflux capacity but not macrophage reverse cholesterol transport. J Lipid Res 58, 731-741
56. Nelson, C. E., Wu, Y., Gemberling, M. P., Oliver, M. L., Waller, M. A., Bohning, J. D., Robinson-Hamm, J. N., Bulaklak, K., Castellanos Rivera, R. M., Collier, J. H., Asokan, A., and Gersbach, C. A. (2019) Long-term evaluation of AAV-CRISPR genome editing for Duchenne muscular dystrophy. Nat Med 25, 427-432
57. Wang, L., Smith, J., Breton, C., Clark, P., Zhang, J., Ying, L., Che, Y., Lape, J., Bell, P., Calcedo, R., Buza, E. L., Saveliev, A., Bartsevich, V. V., He, Z., White, J., Li, M., Jantz, D., and Wilson, J. M. (2018) Meganuclease targeting of PCSK9 in macaque liver leads to stable reduction in serum cholesterol. Nat Biotechnol 36, 717-725
58. Vakulskas, C. A., Dever, D. P., Rettig, G. R., Turk, R., Jacobi, A. M., Collingwood, M. A., Bode, N. M., McNeill, M. S., Yan, S., Camarena, J., Lee, C. M., Park, S. H., Wiebking, V., Bak, R. O., Gomez-Ospina, N., Pavel-Dinu, M., Sun, W., Bao, G., Porteus, M. H., and Behlke, M. A. (2018) A high-fidelity Cas9 mutant delivered as a ribonucleoprotein complex enables efficient gene editing in human hematopoietic stem and progenitor cells. Nat Med 24, 1216-1224
59. Lee, C. M., Davis, T. H., and Bao, G. (2018) Examination of CRISPR/Cas9 design tools and the effect of target site accessibility on Cas9 activity. Exp Physiol 103, 456-460
60. Lee, C. M., Cradick, T. J., Fine, E. J., and Bao, G. (2016) Nuclease Target Site Selection for Maximizing On-target Activity and Minimizing Off-target Effects in Genome Editing. Mol Ther 24, 475-487
61. Lin, Y., Cradick, T. J., Brown, M. T., Deshmukh, H., Ranjan, P., Sarode, N., Wile, B. M., Vertino, P. M., Stewart, F. J., and Bao, G. (2014) CRISPR/Cas9 systems have off-target activity with insertions or deletions between target DNA and guide RNA sequences. Nucleic Acids Res 42, 7473-7485
62. Phillips, M. C. (2014) Apolipoprotein E isoforms and lipoprotein metabolism. IUBMB Life 66, 616-623
63. Lagor, W. R., Fields, D. W., Bauer, R. C., Crawford, A., Abt, M. C., Artis, D., Wherry, E. J., and Rader, D. J. (2014) Genetic manipulation of the ApoF/Stat2 locus supports an important role for type I interferon signaling in atherosclerosis. Atherosclerosis 233, 234-241
64. Michalopoulos, G. K. (2010) Liver regeneration after partial hepatectomy: critical analysis of mechanistic dilemmas. Am J Pathol 176, 2-13
65. Lindblad, B., Lindstedt, S., and Steen, G. (1977) On the enzymic defects in hereditary tyrosinemia. Proc Natl Acad Sci USA 74, 4641-4645
66. Grompe, M. (2001) The pathophysiology and treatment of hereditary tyrosinemia type 1. Semin Liver Dis 21, 563-571
67. Holme, E., and Lindstedt, S. (1998) Tyrosinaemia type I and NTBC (2-(2-nitro-4-trifluoromethylbenzoyl)-1,3-cyclohexanedione). J Inherit Metab Dis 21, 507-517
68. Lindstedt, S., Holme, E., Lock, E. A., Hjalmarson, O., and Strandvik, B. (1992) Treatment of hereditary tyrosinaemia type I by inhibition of 4-hydroxyphenylpyruvate dioxygenase. Lancet 340, 813-817
69. Grompe, M., Lindstedt, S., al-Dhalimy, M., Kennaway, N. G., Papaconstantinou, J., Torres-Ramos, C. A., Ou, C. N., and Finegold, M. (1995) Pharmacological correction of neonatal lethal hepatic dysfunction in a murine model of hereditary tyrosinaemia type I. Nat Genet 10, 453-460
70. Overturf, K., Al-Dhalimy, M., Tanguay, R., Brantly, M., Ou, C. N., Finegold, M., and Grompe, M. (1996) Hepatocytes corrected by gene therapy are selected in vivo in a murine model of hereditary tyrosinaemia type I. Nat Genet 12, 266-273
71. Grompe, M., and Strom, S. (2013) Mice with human livers. Gastroenterology 145, 1209-1214
72. Bissig, K. D., Wieland, S. F., Tran, P., Isogawa, M., Le, T. T., Chisari, F. V., and Verma, I. M. (2010) Human liver chimeric mice provide a model for hepatitis B and C virus infection and treatment. J Clin Invest 120, 924-930
73. Azuma, H., Paulk, N., Ranade, A., Dorrell, C., Al-Dhalimy, M., Ellis, E., Strom, S., Kay, M. A., Finegold, M., and Grompe, M. (2007) Robust expansion of human hepatocytes in Fah−/−/Rag2−/−/Il2rg−/− mice. Nat Biotechnol 25, 903-910
74. Montini, E., Held, P. K., Noll, M., Morcinek, N., Al-Dhalimy, M., Finegold, M., Yant, S. R., Kay, M. A., and Grompe, M. (2002) In vivo correction of murine tyrosinemia type I by DNA-mediated transposition. Mol Ther 6, 759-769
75. Overturf, K., al-Dhalimy, M., Ou, C. N., Finegold, M., Tanguay, R., Lieber, A., Kay, M., and Grompe, M. (1997) Adenovirus-mediated gene therapy in a mouse model of hereditary tyrosinemia type I. Hum Gene Ther 8, 513-521
76. Wang, Z., Lisowski, L., Finegold, M. J., Nakai, H., Kay, M. A., and Grompe, M. (2012) AAV vectors containing rDNA homology display increased chromosomal integration and transgene persistence. Mol Ther 20, 1902-1911
77. Yin, H., Xue, W., Chen, S., Bogorad, R. L., Benedetti, E., Grompe, M., Koteliansky, V., Sharp, P. A., Jacks, T., and Anderson, D. G. (2014) Genome editing with Cas9 in adult mice corrects a disease mutation and phenotype. Nat Biotechnol 32, 551-553
78. Rossidis, A. C., Stratigis, J. D., Chadwick, A. C., Hartman, H. A., Ahn, N. J., Li, H., Singh, K., Coons, B. E., Li, L., Lv, W., Zoltick, P. W., Alapati, D., Zacharias, W., Jain, R., Morrisey, E. E., Musunuru, K., and Peranteau, W. H. (2018) In utero CRISPR-mediated therapeutic editing of metabolic genes. Nat Med 24, 1513-1518
79. Grimm, D., Streetz, K. L., Jopling, C. L., Storm, T. A., Pandey, K., Davis, C. R., Marion, P., Salazar, F., and Kay, M. A. (2006) Fatality in mice due to oversaturation of cellular microRNA/short hairpin RNA pathways. Nature 441, 537-541
80. Paneda, A., Vanrell, L., Mauleon, I., Crettaz, J. S., Berraondo, P., Timmermans, E. J., Beattie, S. G., Twisk, J., van Deventer, S., Prieto, J., Fontanellas, A., Rodriguez-Pena, M. S., and Gonzalez-Aseguinolaza, G. (2009) Effect of adeno-associated virus serotype and genomic structure on liver transduction and biodistribution in mice of both genders. Hum Gene Ther 20, 908-917

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A system, comprising:
(a) a first polynucleotide comprising an expression cassette, said expression cassette comprising a therapeutic polynucleotide linked to an essential gene product polynucleotide, wherein said cassette comprises one or more sequences capable of integrating at least part of the cassette at a first endogenous locus; and
one of (b1) or (b2):
(b1) a second polynucleotide comprising a targeting region capable of inhibiting, knocking down, or disrupting expression of a second endogenous locus and/or the activity of a gene product therefrom,
(b2) a second polynucleotide comprising a targeting region that targets integration at a second endogenous locus to disrupt expression of the second endogenous locus and/or the activity of a gene product therefrom,
wherein for (b1) or (b2) said second endogenous locus encodes the essential gene product in an endogenous form.

2. The system of claim 1, wherein the therapeutic polynucleotide and the essential gene product polynucleotide are linked by a means for co-expression of the therapeutic polynucleotide and the essential gene product polynucleotide.

3. The system of claim 2, wherein the means for co-expression comprises a 2A element or an IRES element.

4. The system of claim 1, wherein in a 5′ to 3′ direction in the expression cassette, the therapeutic polynucleotide is 5′ to the essential gene product polynucleotide.

5. The system of claim 1, wherein in a 5′ to 3′ direction in the expression cassette, the therapeutic polynucleotide is 3′ to the essential gene product polynucleotide.

6. The system of claim 1, wherein the first endogenous locus is the second endogenous locus.

7. The system of claim 1, wherein the essential gene product polynucleotide is fused to the therapeutic polynucleotide.

8. The system of claim 1, wherein the targeting region comprises guide RNA sequence for a CRISPR/Cas9 system.

9. The system of claim 1, wherein the targeting region comprises shRNA, siRNA, anti-sense oligonucleotide, locked nucleic acids, or chemically modified derivatives thereof.

10. The system of claim 1, wherein the first polynucleotide and/or the second polynucleotide serve as a template of integration.

11. The system of claim 1, wherein the first polynucleotide and/or the second polynucleotide are present in a vector.

12. The system of claim 11, wherein the vector comprises a nanoparticle, plasmid, adeno-associated viral vector, lentiviral vector, retroviral vector, or combination thereof.

13. The system of claim 11, wherein the vector is an integrating vector.

14. The system of claim 11, wherein the vector is a non-integrating vector.

15. The system of claim 1, wherein the integration at the first endogenous locus is targeted integration.

16. The system of claim 1, wherein the integration at the first endogenous locus is random integration.

17. The system of claim 1, wherein the expression cassette lacks a promoter.

18. The system of claim 1, wherein integration at the first endogenous locus results in control of expression of the expression cassette from regulatory sequence(s) at the first endogenous locus.

19. The system of claim 18, wherein disruption or reduction of expression at the second endogenous locus that encodes the essential gene product, or disruption of the activity of a gene product therefrom, is therapeutically treatable by one or more nutritional or pharmacological agents to substitute for absence of the essential gene product.

20. The system of claim 1, wherein the essential gene product polynucleotide of claim 1(a) is configured to be resistant to disruption of expression by the targeting region.

21. The system of claim 1, wherein the first endogenous locus is ApoA1 (APOA1), albumin (ALB), haptoglobin (HP), serum amyloid a1 (SAA1), orosomucoid 1 (ORM1), ferritin light chain (FTL), Apolipoprotein C3 (APOC3), fibrinogen beta chain (FGB), fibrinogen gamma chain (FGG), serpin family A member 1 (SERPINA1) or fumarylacetoacetate hydrolase (FAH).

22. The system of claim 1, wherein the essential gene product is fumarylacetoacetate hydrolase (FAH), dehydrodolichyl diphosphate synthase subunit (DHDDS), or 3-hydroxy-3-methylglutaryl Co-enzyme A reductase (HMGCR), UDP glucuronosyltransferase family 1 member A1 (UGT1A1), or methylmalonyl coA mutase (MMUT).

23. The system of claim 1, wherein the pharmacological agent is nitisinone.

24. The system of claim 22, wherein when the essential gene product is DHDDS, cholesterol in the diet of the individual is used for negative selection pressure.

25. The system of claim 22, wherein when the essential gene product is HMGCR, mevalonic acid is used for protection of hepatocytes from selection.

26. The system of claim 1, wherein the system is in vivo in a mammal.

27. The system of claim 26, wherein the mammal is a human.

28. The system of claim 1, wherein the system is ex vivo.

29. A method of effecting gene therapy in an individual, comprising the step of delivering to the individual effective amounts of the first and second polynucleotides of claim 1, said delivering step resulting in selective expansion of cells harboring the therapeutic polynucleotide.

30. The method of claim 23, wherein the second polynucleotide is delivered to the individual prior to, at the same time as, or subsequent to delivery of the first polynucleotide.

31. The method of claim 30, wherein following delivery of the first and second polynucleotides to the individual, expression of the essential gene product is disrupted at the second endogenous locus, and wherein the disruption is therapeutically treatable by delivering to the individual an effective amount of one or more nutritional or pharmacological agents to substitute for absence of the essential gene product.

32. The method of claim 32, wherein the timing of the delivering of the one or more nutritional or pharmacological agents to the individual is dependent on a need of the individual.

33. The method of claim 33, wherein the one or more nutritional or pharmacological agents are delivered to the individual to effect negative selective pressure on cells lacking the first polynucleotides.

34. The method of claim 33, wherein the one or more nutritional or pharmacological agents are delivered to the individual to effect positive selective pressure on cells harboring the polynucleotides.

35. The method of claim 30, wherein the individual has a medical condition related to the therapeutic polynucleotide.

36. The method of claim 30, wherein the individual has a liver medical condition.

37. The method of claim 37, wherein the essential gene product is fumarylacetoacetate hydrolase (Fah), fumarylacetoacetate hydrolase (FAH), dehydrodolichyl diphosphate synthase subunit (DHDDS), or 3-hydroxy-3-methylglutaryl Co-enzyme A reductase (HMGCR), UDP glucuronosyltransferase family 1 member A1 (UGT1A1), ormethylmalonyl coA mutase (MMUT).

38. The method of claim 30, wherein the individual has a urea cycle disorder, branched chain amino acid disorder, amino acid disorder, or inborn error of metabolism with essential liver metabolism.

39. The method of claim 38, wherein when the loss of Fah in cells transfected with the first and second polynucleotides is not needed in the individual, the individual is provided an effective amount of 2-(2-nitro-4-trifluoromethylbenzoyl)-1,3-cyclohexanedione (NTBC).

40. The method of claim 38, wherein when the loss of Fah in cells transfected with the first and second polynucleotides is needed in the individual, the individual is provided an effective amount of a high protein diet.

41. The method of claim 30, wherein the delivering step comprises nanoparticle delivery, transfection, electroporation, hydrodynamic delivery, or a combination thereof.
